(19) United States
(12) Patent Application Publication
(10) Pub. No.: US 2025/0269057 A1
(43) Pub. Date: Aug. 28, 2025

(54) THERAPEUTIC LAMA2 PAYLOAD FOR TREATMENT OF CONGENITAL MUSCULAR DYSTROPHY

(71) Applicant: UNIVERSITAT POMPEU FABRA, Barcelona (ES)

(72) Inventors: Marc GUELL CARGOL, Barcelona (ES); Avencia SANCHEZ MEJAS GARCIA, Barcelona (ES); Maria PALLARES MASMITJA, Barcelona (ES)

(73) Assignee: UNIVERSITAT POMPEU FABRA, Barcelona (ES)

(21) Appl. No.: 18/258,028

(22) PCT Filed: Dec. 16, 2021
(86) PCT No.: PCT/EP2021/086333

§ 371 Date: Jun. 16, 2023

(30) Foreign Application Priority Data
Dec. 16, 2020 (EP) 20214691.6
Nov. 22, 2021 (EP) 21209721.6

(51) Int. Cl. A61K 48/00; A61K 38/17; A61P 21/00; C12N 9/12; C12N 9/22; C12N 15/11; C12N 15/86; C12N 15/88; C12N 15/90

(52) U.S. Cl. CPC A61K 48/005; A61K 38/1709; A61P 21/00; C12N 9/1241; C12N 9/226; C12N 15/111; C12N 15/86; C12N 15/88; C12N 15/907; C07K 2319/80; C12N 2310/20; C12N 2740/15043; C12N 2830/50; C12Y 207/07

(57) ABSTRACT

The present invention relates to a composition comprising:
a) a first protein comprising or consisting of a site-specific DNA binding protein capable of binding and cleaving a target nucleic acid sequence; or a nucleic acid construct encoding said first protein;
b) a second protein comprising or consisting of a transposase; or a nucleic acid construct encoding said second protein; and
c) a nucleic acid construct comprising a transgene encoding laminin-α2 protein, or a functional variant or fragment thereof.

It also relates to the therapeutic use of this composition, to integrate a LAMA2 transgene into a specific site within the genome of a cell, in particular for the treatment of congenital muscular dystrophy.

### TECHNICAL FIELD

The present invention relates to a composition comprising a fusion protein comprising a site-specific DNA-binding protein fused to a transposase and LAMA2 transgene to integrate LAMA2 transgene into a specific site within the genome of a cell and its therapeutic use, in particular for the treatment of congenital muscular dystrophy.

### BACKGROUND

Congenital muscular dystrophy (CMD) is a class of severe early-onset muscular dystrophies affecting skeletal/cardiac muscles as well as the central nervous system.

Among CMD, laminin-α2 chain-deficient co-genital muscular dystrophy (LAMA2 MD), also known as merosin-deficient congenital dystrophy type 1A (MDCIA), is characterized by severe hypotonia, muscle weakness, skeletal deformity, non-ambulation, and respiratory insufficiency. There are milder forms of the disease with later-onset, with similar symptoms but wilder phenotypic variability.

There are currently no cure available for MDCIA. Current strategies in the clinic are focused on management (feeding supplementation, non-invasive ventilation support for respiratory insufficiency, and physical therapy for joint contractures, spinal defects, and other issues) (Nguyen et al., 2019. Appl Clin Genet. 12:113-130).

MDC1A is caused by loss-of-function recessive mutations in both copies of the LAMA2 gene coding for the laminin-α2 chain, one of the subunits of laminin-211. Laminin-211 is an extracellular matrix protein that functions to stabilize the basement membrane and muscle fibers during contraction. The laminin-α2 chain protein is 3 122 amino acid residue long, hence its coding DNA sequence (CDS) is ˜9.3 kb long. As with many other muscular dystrophies, MDCIA is therefore caused by mutations in a very large gene, surpassing the cargo capacity of AAV vectors or lentiviral vectors. Moreover, several tens of different mutations have been described so far in the overall LAMA2 gene, from single point missense, splice site or in-frame mutations to multi-kilobase region deletions (Oliveira et al., 2018. Hum Mutat. 39(10):1314-1337), so much so that a curative gene therapeutic approach is challenging, and gene replacement strategies are not available this far.

Other corrective gene-based strategies have been tested for MDC1A. Exon-skipping approach, which commonly refers to the use of synthetic antisense oligonucleotide to inhibit a splice enhancer site and prevent a particular exon from participating in splicing, was used (Aoki et al., 2013. Biomed Res Int. 2013:402369). However, this exon-skipping approach is not universal and can only be used with some patients that have a deletion, where the reading frame could be restored by skipping an additional exon adjacent to the deletion (Shieh, 2018. Neurotherapeutics. 15(4):840-848). Exon-skipping restores translation of truncated but partially functional proteins but does not recapitulate the benefit of the full-length laminin-α2 protein.

CRISPR/Cas9 was also used to correct LAMA2 mutation, in particular to excise the LAMA2 intron 2 region containing a splice-site mutation and creating a functional donor splice site, through non-homologous end-joining (NHEJ), leading to inclusion of exon 2 of the LAMA2 transcript and restoration of full-length laminin-α2 chain protein (Kemaladewi et al., 2017. Nat Med. 23(8):984-989). As exon-skipping, this strategy can only be used in some patients and is not universal.

However, NHEJ is not suitable for integrating large transgenes into a genome, and, while gene modification relying on the homology directed repair (HDR) pathway can theoretically correct the majority of pathogenic point mutations, it was found to be “extremely inefficient”, as explained by Kemaladewi & Cohn (2019. Emerg Top Life Sci. 3(1):11-18). In conclusion, Kemaladewi & Cohn called for the development of alternative strategies to correct mutations causing muscular dystrophy.

Finally, CRISPR/Cas9 was also used to induce gene expression, by using a catalytically inactive Cas9 (SadCas9) fused to a transcription activation domain, such as VP64, which is directed to the promoter of the LAMA1 gene, thereby allowing an increased expression of the structurally similar laminin-al chain protein (Kemaladewi et al., 2019. Nature. 572 (7767): 125-130). However, this approach needs a continuous expression of the transgene.

There remains thus a need to develop a permanent and efficient strategy for treating MDC1A, that is universal and not only limited to certain patients affected with certain mutations.

### SUMMARY

Gene replacement of the LAMA2 gene has promising therapeutic value; however, it has been challenging so far due to cargo size constrains. The Inventors used a fusion protein comprising a site-specific DNA-binding protein (e.g., Cas9) fused to a transposase (e.g., a hyperactive PiggyBac transposase) to integrate a healthy full-length copy of the LAMA2 gene into a specific site within the genome of a cell. The Inventors have thereby been able to provide a therapeutic payload with a large LAMA2 expression cassette (9.3 kb of the CDS) for ex vivo and in vivo delivery, compatible with different gene delivery technologies.

Hence, the present invention relates to a composition comprising a) a first protein comprising or consisting of a site-specific DNA binding protein capable of binding and cleaving a target nucleic acid sequence; or a nucleic acid construct encoding said first protein; b) a second protein comprising or consisting of a transposase; or a nucleic acid construct encoding said second protein; and c) a nucleic acid construct comprising a transgene encoding laminin-α2 protein, preferably of SEQ ID NO: 74, or a functional variant or fragment thereof.

In one embodiment, the first and second proteins of a) and b) are fused together, optionally through a linker.

In one embodiment, the nucleic acid construct of c) comprises a promoter selected from the group consisting of: CMV promoter of SEQ ID NO: 76, CAG promoter of SEQ ID NO: 77, EF1-alpha promoter of SEQ ID NO: 78, SV-40 promoter of SEQ ID NO: 79 and EalbAAT promoter of SEQ ID NO: 80. In one embodiment, the nucleic acid construct of c) comprises a splice acceptor of SEQ ID NO: 81. In one embodiment, the nucleic acid construct of c) comprises a poly(A) signal sequence, preferably selected from the group consisting of SEQ ID NO: 83-85 and/or an insulator element, preferably selected from the group consisting of SEQ ID NO: 86-87. In one embodiment, the nucleic acid construct of c) is flanked by inverted terminal repeats (ITR), preferably 5′-ITR and 3′-ITR of SEQ ID NO: 88 and 89.

In one embodiment, the nucleic acid construct of c) is comprised in a vector selected from the group consisting of: plasmid vector, a minicircle vector, a doggy bone DNA donor vector, a lentivirus vector and retrovirus vector.

In one embodiment, said site-specific DNA binding protein is RNA-guided nuclease comprising a Cas protein, and wherein said composition further comprises a guide RNA including a complementary sequence to a target nucleic acid sequence for integrating said LAMA2 transgene in a specific site of a genome of cell, preferably said Cas protein is S. pyogenes Cas 9 protein.

In one embodiment, the guide RNA comprises any one of SEQ ID NOs: 90 to 97.

In one embodiment, said transposase is a modified hyperactive Piggybac transposase or Sleepy Beauty transposase, preferably a modified hyperactive PiggyBac transposase comprising one or more amino acid mutations to increase excision activity as compared to unmodified hyperactive Piggybac, and one or more amino acid mutations to decrease DNA binding activity as compared to unmodified hyperactive Piggybac.

In one embodiment, said hyperactive PiggyBac transposase is a modified hyperactive PiggyBac transposase comprising at least one mutation of amino acid selected from the group consisting of: V34, T43, Y177, M194, R202, S230, R245, R275, R277, G325, S351, N347, R372, K375, R376, E377, E380, A411, D450, T560, S564, S573, M589, S592, and F594 preferably comprising mutations of amino acids Y177, R202, S230, R245, R275, R277, G325, N347, S351, E377, D450, R372 and K375, E377, T560, S564, S573, M589, S592, F594, said position number corresponding to the amino acid number of unmodified hyperactive Piggybac of SEQ ID NO: 9. In one embodiment, said hyperactive PiggyBac transposase is a modified hyperactive PiggyBac transposase comprising at least one mutation of amino acid selected from the group consisting of: M194, R245, R275, R277, G325, R372, K375, R376, E377, E380, D450 and S573, preferably comprising mutations of amino acids D450, R372 and R375, said position number corresponding to the amino acid number of unmodified hyperactive Piggybac of SEQ ID NO: 9.

In one embodiment, said transposase is fused N-terminally to said site-specific DNA binding protein by a linker, preferably a peptidic linker comprising GGS, XTEN or FOKI, more preferably XTEN of SEQ ID NO: 53.

In one embodiment, said composition is packaged within a nanoparticle.

The present invention also relates to an in vitro method for integrating LAMA2 transgene into a target nucleic acid sequence within the genome of a cell comprising introducing into a cell the composition of the invention.

The present invention also relates to an engineered cell obtainable by the in vitro method of the invention, wherein the engineered cell comprises a nucleic acid integrated within its genome, said nucleic acid comprising a transgene encoding laminin-α2 protein flanked by operational sequences for integrase and/or transposase mediated gene insertion.

In one embodiment, the operational sequences flanking the transgene comprise or consists of SEQ ID NO: 88 and 89.

In one embodiment, the inter-ITR size is at least 300 bp.

In one embodiment, the transgene encodes the full-length laminin-α2 protein.

The present invention also relates to a pharmaceutical composition comprising the composition of the invention, or the engineered cell of the invention, optionally in combination with one or more pharmaceutically acceptable excipients.

The present invention also relates to the composition of the invention, the engineered cell of the invention or the pharmaceutical composition of the invention, for use in therapy, in particular for use in the treatment of merosin-deficient congenital muscular dystrophy type 1A (MDC1A) in a subject in need thereof.

### Definitions

As used herein, the singular forms “a”, “an”, and “the” include the singular and the plural reference unless the context clearly indicates otherwise. Thus, for example, a reference to “an agent” includes a single agent and a plurality of such agents.

The terms “nucleic acid sequence” and “nucleotide sequence” may be used interchangeably to refer to any molecule composed of, or comprising, monomeric nucleotides. A nucleic acid may be an oligonucleotide or a polynucleotide. A nucleotide sequence may be a DNA, RNA, or a mix thereof. A nucleotide sequence may be chemically-modified or artificial. Nucleotide sequences include peptide nucleic acids (PNA), morpholinos and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include, without limitation, methylphosphonates, phosphoramidates, phosphorodithioates, N3′P5′-phosphoramidates and oligoribonucleotide phosphorothioates and their 2′-O-allyl analogs and 2′-O-methylribonucleotide methylphosphonates which may be used in a nucleotide of the disclosure.

The term “transgene” refers to an exogenous nucleic acid sequence, in particular an exogenous DNA or cDNA encoding a gene product. The gene product may be an RNA, peptide or protein. In addition to the coding region for the gene product (CDS), the transgene may include or be associated with one or more operational sequences to facilitate or enhance expression, such as a promoter, enhancer(s), response element(s), reporter element(s), insulator element(s), polyadenylation signal(s) and/or other functional elements. Embodiments of the disclosure may utilize any known suitable promoter, enhancer(s), response element(s), reporter element(s), insulator element(s), polyadenylation signal(s) and/or other functional elements, unless specified otherwise. Suitable elements and sequences will be well known to those skilled in the art.

The term “nucleic acid construct” refers to a man-made nucleic acid molecule resulting from the use of recombinant DNA technology. A nucleic acid construct is a nucleic acid molecule, either single- or double-stranded, which has been modified to contain segments of nucleic acids sequences, which are combined and juxtaposed in a manner, which would not otherwise exist in nature. A nucleic acid construct usually is a “vector”, i.e., a nucleic acid molecule which is used to deliver exogenously created DNA into a host cell.

The term “vector” or “expression vector” may refers either to an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome, which may contain any means for assuring self-replication; or to an integrative vector, i.e., a vector that, when introduced into a host cell, is integrated into its genome and replicated together with the chromosome(s) into which it has been integrated.

The terms “sequence identity” or “identity” refers to the number (%) of matches (identical amino acid residues or nucleotides) in positions from an alignment of two polypeptide or polynucleotide sequences. The sequence identity is determined by comparing the sequences when aligned so as to maximize overlap while minimizing sequence gaps. In particular, sequence identity may be determined using any of a number of mathematical global or local alignment algorithms, depending on the length of the two sequences. Sequences of similar lengths are preferably aligned using a global alignment algorithm (e.g., Needleman and Wunsch algorithm [Needleman & Wunsch, 1970. J Mol Biol. 48(3):443-53]) which aligns the sequences optimally over the entire length, while sequences of substantially different lengths are preferably aligned using a local alignment algorithm (e.g., Smith and Waterman algorithm [Smith & Waterman, 1981. J Mol Biol. 147(1):195-197] or Altschul algorithm [Altschul et al., 1997. Nucleic Acids Res. 25(17):3389-3402; Altschul et al., 2005. FEBS J. 272(20):5101-9]). Alignment for purposes of determining percent amino acid sequence identity can be achieved in various ways that are within the skill in the art, for instance, using publicly available computer software available on internet web sites such as http://blast.ncbi.nlm.nih.gov/or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleotide or amino acid sequence identity values refers to values generated using the pair wise sequence alignment program EMBOSS Needle that creates an optimal global alignment of two sequences using the Needleman-Wunsch algorithm, wherein all search parameters are set to default values, i.e., Scoring matrix=BLOSUM62, Gap open=10, Gap extend=0.5, End gap penalty=false, End gap open=10 and End gap extend=0.5.

The term “fusion” refers to a molecule in which two or more subunit molecules are linked. In some embodiments, the link between the two is covalent; alternatively, the link between the two can be non-covalent and rely, e.g., on intermolecular interactions. The subunit molecules can be the same chemical type of molecule, or can be different chemical types of molecules.

The term “fusion protein” refers to a hybrid polypeptide which comprises protein domains from at least two different proteins. For example, one protein domain may be located at the amino-terminal (N-terminal) portion of the fusion protein or at the carboxy-terminal (C-terminal) protein, thus forming an “amino-terminal fusion protein” or a “carboxy-terminal fusion protein”, respectively. In preferred embodiments, a fusion protein is a single chain polypeptide which may be fully encoded by a nucleic acid sequence, and includes at least two protein domains directly covalently linked by peptidic bound or optionally covalently linked via a peptidic linker.

The term “linker” refers to a chemical group or a molecule linking two adjacent molecules or moieties.

The term “binding protein” refers to a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to one or more molecules of the same protein to form homodimers, homotrimers, etc.; and/or it can bind to one or more molecules of a different protein or proteins. A binding protein can have more than one type of binding activity. For example, zinc finger proteins have DNA-binding, RNA-binding and protein-binding activity.

The terms “Cas9” or “Cas9 nuclease” refer to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA” or simply “gNRA”) can be engineered so as to incorporate aspects of both the crRNA and tracrRNA into a single RNA species.

Cas9 recognizes a short motif in the CRISPR repeat sequences (the PAM or protospacer adjacent motif) to help distinguish self vs. non-self. Cas9 nuclease sequences and structures are well known to those of skill in the art. Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., 2013. (RNA Biol. 10(5):726-37), the entire content of which is incorporated herein by reference.

In some embodiments, a Cas9 nuclease has an inactive (e.g., an inactivated) DNA cleavage domain. A nuclease-inactivated Cas9 protein can interchangeably be referred to as a “dCas9” protein (for nuclease-“dead” Cas9). Methods for generating a Cas9 protein (or a fragment thereof) having an inactive DNA cleavage domain are known in the art (see, e.g., Jinek et al., 2012. Science. 337(6096):816-821; Qi et al., 2013. Cell. 152(5):1173-83, the entire content of each being incorporated herein by reference).

The term “zinc finger protein” refers to a protein, or a domain within a larger protein, that binds DNA in a sequence-specific manner through one or more zinc fingers, which are regions of amino acid sequences within a binding domain of the zinc finger protein whose structure is stabilized through coordination of a zinc ion. The term “zinc finger protein” is often abbreviated as “ZFP”.

The term “zinc finger nuclease” refers to an artificial restriction enzyme generated by fusing a zinc finger DNA-binding domain to a DNA-cleavage domain. Zinc finger domains can be engineered to target specific desired DNA sequences, and this enables zinc finger nucleases to target unique sequences within complex genomes. “Zinc finger nuclease” is often abbreviated as “ZFN” or “ZNP”.

The term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

The term “specificity” refers to the ability to selectively bind a sequence which shares a degree of sequence identity to a selected sequence.

The terms “insertion” and “integration” refer to the addition of a nucleic acid sequence into a second nucleic acid sequence or into a genome or part thereof. The terms “specific”, “site-specific”, “targeted” and “on-targeted” in relation to insertion or integration, are used herein interchangeably to refer to the insertion of a nucleic acid into a specific site of a second nucleic acid or into a specific site of a genome or part thereof. Conversely, the terms “random”, “non-targeted” and “off-targeted” refer to non-specific and unintended insertion of a nucleic acid into an unwanted site. The terms “total” or “overall” refer to the total number of insertions.

The term “mutation” refers to a substitution of a residue within a sequence, e.g., a nucleic acid or amino acid sequence, with another residue; and/or to a deletion or insertion of one or more residues within a nucleic acid or amino acid sequence. Mutations are typically described herein by identifying the original residue followed by the position of the residue within the sequence, then the identity of the newly substituted residue. Various methods for making amino acid substitutions (mutations) provided herein are well known in the art, and are provided by, for example, Green & Sambrook, 2012 (Molecular cloning: a laboratory manual (4th Ed.). Cold Spring Harbor Laboratory Press, Cold Spring Harbor, N.Y.). In preferred embodiments, the term mutation in a protein refers to an amino acid substitution.

The term “transposase” refers to an enzyme that binds to the end of a transposon and catalyzes its movement to another part of the genome by a cut-and-paste mechanism or a replicative transposition mechanism.

The term “modified” refers to a protein or nucleic acid sequence that is different than a corresponding unmodified protein or nucleic acid sequence.

The term “linker” refers to a chemical group or a molecule linking two adjacent molecules or moieties.

### DETAILED DESCRIPTION

The inventors used a fusion protein comprising a site-specific DNA-binding protein fused to a transposase to integrate LAMA2 transgene into a specific site within the genome of a cell.

The present invention relates thus to a composition comprising or consisting of:
a) a first protein comprising or consisting of a site-specific DNA binding protein capable of binding and cleaving a target nucleic acid sequence; or a nucleic acid construct, preferably a cDNA or an mRNA, encoding said first protein;
b) a second protein comprising or consisting of a transposase; or a nucleic acid construct, preferably a cDNA or an mRNA, encoding said second protein; and
c) a nucleic acid construct comprising or consisting of a transgene encoding the laminin-α2 protein, functional variant or fragment thereof.

In one embodiment, the composition comprises or consists of:
a) a fusion protein comprising or consisting of (i) a first protein comprising or consisting of a site-specific DNA binding protein capable of binding and cleaving a target nucleic acid sequence, and (ii) a second protein comprising or consisting of a transposase; or a nucleic acid construct, preferably a cDNA or an mRNA encoding said fusion protein; and
b) a nucleic acid construct comprising or consisting of a transgene encoding the laminin-α2 protein, functional variant or fragment thereof.

According to the invention, the first protein comprises or consists of a site-specific DNA binding protein capable of binding and cleaving a target nucleic acid sequence.

Current genome engineering tools, including engineered zinc finger proteins (ZFPs), transcription activator like effector nucleases (TALENs), and more recently, the RNA-guided DNA nucleases such as Cas9, effect sequence-specific DNA cleavage in a genome. This programmable cleavage can result in mutation of the DNA at the cleavage site via non-homologous end joining (NHEJ) or replacement of the DNA surrounding the cleavage site via homology-directed repair (HDR).

In one embodiment, the site-specific DNA binding protein is selected from the group comprising or consisting of RNA-guided DNA nucleases, zinc finger proteins and transcription activator like effector nucleases.

In one embodiment, the site-specific DNA binding protein is selected from the group comprising or consisting of RNA-guided DNA nucleases and zinc finger proteins.

In one embodiment, the site-specific DNA binding protein is an RNA-guided nuclease.

In one embodiment, the site-specific DNA binding protein is a Cas9 protein (e.g., without limitation, Streptococcus pyogenes Cas9 (SpCas9), Staphylococcus aureus Cas9 (SaCas9), or Campylobacter jejuni Cas9 (CjCas9); some other suitable examples will be described below), or a variant thereof (e.g., nickase Cas9 (nCas9) or dead Cas9 (dCas9)), a Cas12a protein, a Cas12b protein, a Cpf1 protein, or a CasX protein, including variants and functional fragments thereof.

In one embodiment, the site-specific DNA binding protein is a Cas9 protein, including variants and functional fragments thereof.

The CRISPR-Cas9 system is a highly effective tool for inactivating or modifying genes via sequence-specific double-strand breaks (DSBs). These DSBs are recognized by the cellular DNA damage response machinery and can be repaired by endogenous DSB repair pathways. The predominant repair pathway is non-homologous end joining (NHEJ), which often results in small insertions and/or deletions that can create frameshift mutations and disrupt the function of genes. This pathway can be exploited to generate genetic knockout mutations. Alternatively, in the presence of repair templates (such as, e.g., the nucleic acid construct comprising or consisting of a transgene encoding the laminin-α2 protein, functional variant or fragment thereof), the damage can be repaired seamlessly by homology-directed repair (HDR). However, despite remarkable progress, HDR-mediated genome editing to introduce precise genetic modifications is much less efficient than NHEJ-mediated gene disruption. Furthermore, large multi-kb replacements by the HDR pathways results challenging and requires selection and/or large population cell sorting. Consequently, the major applications for the HDR pathways are currently limited to the local replacement of key regions within genes, but not of large, full-length genes. As explained above, the present invention remedies this deficiency.

In one embodiment, the Cas9 protein comprises (i) an active DNA cleavage domain and (ii) a guide RNA binding domain.

Among the known Cas9 proteins, the S. pyogenes Cas9 protein has been widely used as a tool for genome engineering. This Cas9 protein is a large, multi-domain protein containing two distinct nuclease domains.

In one embodiment, the Cas9 protein is selected from the group comprising or consisting of the Cas9 protein from Corynebacterium ulcerans (NCBI Refs: NC_015683.1, NC_017317.1) with SEQ ID NO: 19); Corynebacterium diphtheria (NCBI Refs: NC_016782.1, NC_016786.1) with SEQ ID NO: 20; Spiroplasma syrphidicola (NCBI Ref: NC_021284.1) with SEQ ID NO: 21; Prevotella intermedia (NCBI Ref: NC_017861.1) with SEQ ID NO: 22; Spiroplasma taiwanense (NCBI Ref: NC_021846.1) with SEQ ID NO: 23; Streptococcus iniae (NCBI Ref: NC_021314.1) with SEQ ID NO: 24; Belliella baltica (NCBI Ref: NC_018010.1) with SEQ ID NO: 25; Psychroflexus torquisi (NCBI Ref: NC_018721.1) with SEQ ID NO: 26; Streptococcus thermophilus (NCBI Ref: YP_820832.1) with SEQ ID NO: 27; Listeria innocua (NCBI Ref: NP_472073.1) with SEQ ID NO: 28; Campylobacter jejuni (CjCas9) (NCBI Ref: YP_002344900.1) with SEQ ID NO: 29 (encoded by SEQ ID NO: 63); Neisseria meningitidis (NCBI Ref: YP_002342100.1) with SEQ ID NO: 30; Staphylococcus aureus (SaCas9) with SEQ ID NO: 68 (encoded by SEQ ID NO: 60); and Streptococcus pyogenes (SpCas9) (NCBI Ref: NC_017053.1) with SEQ ID NO: 31.

In one embodiment, when referring herein to the wild-type Cas9 protein, said wild-type Cas9 protein corresponds to Cas9 from Streptococcus pyogenes (spCas9) with SEQ ID NO: 31, unless specified otherwise.

In one embodiment, the Cas9 protein may be a “Cas9 variant”. A “Cas9 variant”, as used herein, is a protein sharing homology to a Cas9 protein as described herein, and includes fragments thereof.

In one embodiment, the Cas9 variant can be at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild-type Cas9 protein with SEQ ID NO: 31, or to any other Cas9 protein with SEQ ID NOs: 19-30 or 68.

In one embodiment, the Cas9 variant comprises the amino acid sequence of a Cas9 protein with one or several amino acid substitutions. For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand.

Mutations within these subdomains can silence the nuclease activity of Cas9. For example, the substitutions D10A and H841A are known to completely inactivate the nuclease activity of the S. pyogenes Cas9 protein with SEQ ID NO: 31, resulting in a dead Cas9 (dCas9) that still retains its ability to bind DNA in a sgRNA-programmed manner. In principle, when fused to another protein or domain, dCas9 can target that protein to virtually any DNA sequence simply by co-expression with an appropriate sgRNA. In one embodiment, the dCas9 protein is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 59. In one embodiment, the dCas9 protein comprises or consists of an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 67.

As to Cas9 nickase (nCas9), it is a variant of Cas9 nuclease differing by a point mutation (D10A) in the RuvC nuclease domain, which enables it to nick, but not cleave, DNA. In one embodiment, the nCas9 protein is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 57. In one embodiment, the nCas9 protein comprises or consists of an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 65. In some embodiments, the SaCas9 nickase (SanCas9) is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 58. In some embodiments, the SaCas9 nickase (SanCas9) comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 66.

In one embodiment, the Cas9 variant comprises a fragment of Cas9, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of a wild-type Cas9 protein with SEQ ID NO: 31, or of any other Cas9 protein with SEQ ID NOs: 19-30 or 68.

In one embodiment, the Cas9 variant comprises only one of a DNA cleavage domain or a guide RNA binding domain.

In one embodiment, an exemplary Cas9 variant is humanized Cas9 (hCas9) or a variant or functional fragment thereof. As used herein, the term “humanized Cas9” or “hCas9” refers to a sequence-optimized Cas9 protein for human cells.

In one embodiment, the hCas9 protein is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 56. In one embodiment, the hCas9 protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 64.

In one embodiment, the site-specific DNA binding protein is a cpf1 protein. In one embodiment, the cpf1 protein is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 61. In one embodiment, the cpf1 protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 69.

In one embodiment, the site-specific DNA binding protein is a CasX protein. In one embodiment, the CasX is encoded by a nucleic acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 62. In one embodiment, the CasX comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 70.

As will be further detailed below, certain aspects of the disclosure are also directed to vectors or plasmids (e.g., expression vectors, packaging vectors, etc.) comprising a nucleic acid construct encoding the site-specific DNA binding protein, in particular the RNA-guided nuclease, in particular any of the Cas9 proteins described herein; said vectors or plasmids being preferably suitable for expression in a host cell, e.g., mammalian cells, yeast cells, insect cells, plant cells, fungal cells, or algal cells.

In one embodiment, the site-specific DNA binding protein is a zinc finger protein (ZFP).

Zinc finger proteins are proteins that can bind to DNA in a sequence-specific manner. ZFP are unevenly distributed in eukaryotes. ZFP have been identified that are involved in DNA recognition, RNA binding, and protein binding. Certain classifications for zinc finger proteins are based on “fold groups” in view of the overall shape of the protein backbone in the folded domain. The most common “fold groups” of zinc fingers are the C2H2 or Cys2His2-like (the “classic zinc finger”), treble clef, and zinc ribbon. Representative motifs characterizing these proteins are disclosed in Table 1 of Li & Liu, 2020 (Int J Mol Sci. 21(4):1361), which Table is herein incorporated by reference.

The ZFP can be any ZFP, variant or functional fragment thereof, that can bind to a specific genomic DNA sequence in a genome. Non-limiting examples of ZFPs include ZFPs comprising a fold group or zinc finger motif selected from C2H2, gag knuckle, treble clef, zinc ribbon, Zn2/Cys6-like, or TAZ2 domain-like, or any combination thereof. In one embodiment, the ZFP is a C2H2 zinc finger protein.

In one embodiment, the ZFP is an engineered ZFP. Engineered zinc finger arrays can be fused to a DNA cleavage domain (usually the cleavage domain of FokI) to generate zinc finger nucleases. Such zinc finger-FokI fusions have become useful reagents for manipulating genomes.

The ZFP can comprise 2, 3, 4, 5, 6, 7, 8, 9, 10, 11, 12, or more zinc finger domains. The ZFP can comprise from 2 to 12, from 2 to 10, from 2 to 8, from 3 to 8, from 4 to 8, or from 5 to 8 zinc finger domains. In one embodiment, the ZFP comprises 6 zinc finger domains.

A common modular assembly process involves combining separate zinc fingers that can each recognize a 3-basepair DNA sequence to generate 3-finger, 4-, 5-, or 6-finger arrays that recognize target sites ranging from 9 basepairs to 18 basepairs in length. Another method uses 2-finger modules to generate zinc finger arrays with up to six individual zinc fingers.

In one embodiment, the binding domain of the ZFP can be engineered to bind to a sequence of interest. An engineered zinc finger binding domain can have improved binding specificity, compared to a naturally-occurring ZFP.

In one embodiment, exemplary nucleic acid sequences encoding the ZFP comprise or consists of SEQ ID NO: 32, SEQ ID NO: 34, SEQ ID NO: 36, or SEQ ID NO: 38. In one embodiment, exemplary amino acid sequences encoded by these sequences comprise or consists of SEQ ID NO: 33, SEQ ID NO: 35, SEQ ID NO: 37, or SEQ ID NO: 39.

In one embodiment, the ZFP comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to any one of SEQ ID NOs: 33, 35, 37 or 39.

In one embodiment, the ZFP does not have a Gal4 DNA binding domain. Gal4 binds to CGG-N₁₁-CCG, where N can be any base. This protein is a positive regulator for the gene expression of the galactose-induced genes such as GAL1, GAL2, GAL7, GAL10, and MEL1 which code for the enzymes used to convert galactose to glucose. It recognizes a 17-base pair sequence in the upstream activating sequence (UAS-G) of these genes. Therefore, Gal4 recognizes a short and very frequent sequence in the genome, thus not being site-specific. In one embodiment, the ZFP has a Gal4 DNA binding domain engineered to be site-specific.

As will be further detailed below, certain aspects of the disclosure are directed to vectors or plasmids (e.g., expression vectors, packaging vectors, etc.) comprising a nucleic acid construct encoding the site-specific DNA binding protein, in particular the ZFP described herein; said vectors or plasmids being preferably suitable for expression in a host cell, e.g., mammalian cells, yeast cells, insect cells, plant cells, fungal cells, or algal cells.

According to the invention, the second protein comprises or consists of a transposase.

Transposons are chromosomal segments that can undergo transposition, e.g., DNA that can be translocated as a whole in the absence of a complementary sequence in the host DNA. Transposons can be used to perform long-range DNA engineering in human cells. Common transposon systems used in mammalian cells include, without limitation, Sleeping Beauty (SB), which was reconstructed from inactive transposons, and PiggyBac (PB), isolated from the moth Trichoplusia. PiggyBac has higher transposition activity than SB and it can be excised scarlessly.

Native DNA transposons typically contain a single gene coding for a transposase protein, which is flanked by Inverted Terminal Repeats (ITRs) that carry transposase binding sites. During their transposition, the transposase protein recognizes these ITRs to catalyze excision and subsequent reintegration of the element elsewhere in a random manner. Moreover, some of these transposons can be adapted for use in gene therapy protocols, employing them as bi-component systems, in which a plasmid contains an expression cassette where a DNA sequence of interest (e.g., a LAMA2 transgene), placed between the transposon ITRs, can be introduced into a host genome directed by a co-transfected plasmid containing the sequence encoding the transposase enzyme or its mRNA synthesized in vitro. According to the disclosure, a transposon-based system is used to efficiently mediate stable integration and persistent expression of LAMA2 transgene in a cell.

In one embodiment, a transposase or modified transposase used in the present invention can be any transposase capable of inserting a LAMA2 transgene into a specific site of a genome.

Non-limiting examples of transposases include Frog Prince, Sleeping Beauty, hyperactive Sleeping Beauty, PiggyBac, and hyperactive PiggyBac.

In one embodiment, the transposase is a hyperactive PiggyBac transposase (hyPB).

The wild-type hyperactive PiggyBac transposase has an amino acid sequence comprising or consisting of SEQ ID NO: 9. An exemplary nucleic acid sequence coding this protein is as set forth in SEQ ID NO: 71.

In one embodiment, the transposase is a modified hyperactive PiggyBac transposase.

As used herein, “modified hyperactive PiggyBac transposase” refers to a transposase comprising one or more amino acid substitutions, typically no more than 1, 2, 3, 4, 5, 6, 7, 8, 9, or 10 amino acid substitutions, as compared to the wild-type hyperactive PiggyBac transposase with SEQ ID NO: 9. More specifically, a modified hyperactive PiggyBac comprises (i) one or more amino acid substitutions to increase excision activity as compared to the wild-type hyperactive PiggyBac transposase, and/or (ii) one or more amino acid substitutions to decrease DNA binding activity as compared to the wild-type hyperactive PiggyBac transposase. In one embodiment, the modified hyperactive PiggyBac transposase comprises an amino acid sequence at least 85%, at least 90%, at least 95%, at least 96%, at least 97%, at least 98%, or at least 99% identical to the sequence set forth in SEQ ID NO: 9.

In some embodiment, the modified hyperactive PiggyBac transposase comprises one or more amino acid substitutions, as compared to the wild-type hyperactive PiggyBac transposase, to increase excision activity.

In one embodiment, the modified hyperactive PiggyBac transposase comprises one or more amino acid substitutions to increase excision activity within regions defined by the amino acid position numbers [194-200], [214-222], [434-442] and/or [446-456], for example at amino acid positions D198, D201, R202, M212, and/or S213; numbering based on the wild-type hyperactive PiggyBac transposase with SEQ ID NO: 9.

In one embodiment, the modified hyperactive PiggyBac transposase comprises one or more amino acid substitutions to increase excision activity at amino acid positions selected from positions 450, 560, 564, 573, 589, 592, and/or 594; numbering based on the wild-type hyperactive PiggyBac transposase with SEQ ID NO: 9.

In one embodiment, the modified hyperactive PiggyBac transposase comprises one or more amino acid substitutions to increase excision activity at amino acid positions selected from positions M194 and D450; numbering based on the wild-type hyperactive PiggyBac transposase with SEQ ID NO: 9. In one embodiment, the one or more amino acid substitutions are selected among M194V and D450N, numbering based on the wild-type hyperactive PiggyBac transposase with SEQ ID NO: 9.

In one embodiment, the modified hyperactive PiggyBac transposase comprises one or more amino acid substitutions, as compared to the wild-type hyperactive PiggyBac transposase, to decrease DNA binding activity.

In one embodiment, the modified hyperactive PiggyBac transposase comprises one or more amino acid substitutions to decrease DNA binding activity at amino acid positions selected from positions 254, 275, 277, 347, 372, 375, and/or 465; numbering based on the wild-type hyperactive PiggyBac transposase with SEQ ID NO: 9.

In one embodiment, the modified hyperactive PiggyBac transposase comprises one or more amino acid substitutions to decrease DNA binding activity at amino acid positions selected from positions R275, N347, R372, K375, R376, E377, and/or E380; numbering based on the wild-type hyperactive PiggyBac transposase with SEQ ID NO: 9.

In one embodiment, the modified hyperactive PiggyBac transposase comprises one or more amino acid substitutions to decrease DNA binding activity at amino acid positions selected from positions R372, K375, R376, E377, and/or E380; numbering based on the wild-type hyperactive PiggyBac transposase with SEQ ID NO: 9. In one embodiment, the one or more amino acid substitutions are selected among R372A, K375A, R376A, E377A, and/or E380A; numbering based on the wild-type hyperactive PiggyBac transposase with SEQ ID NO: 9.

In one embodiment, the modified hyperactive PiggyBac transposase comprises one or more amino acid substitutions to decrease DNA binding activity at amino acid positions selected from positions N347, R372, and K375; numbering based on the wild-type hyperactive PiggyBac transposase with SEQ ID NO: 9. In one embodiment, the one or more amino acid substitutions are selected among N347S, N347A, R372A and/or K375A; numbering based on the wild-type hyperactive PiggyBac transposase with SEQ ID NO: 9. In one embodiment, the one or more amino acid substitutions are selected among N347S or N347A; numbering based on the wild-type hyperactive PiggyBac transposase with SEQ ID NO: 9.

In one embodiment, the modified hyperactive PiggyBac transposase comprises one or more amino acid substitutions to increase excision activity, as defined above; and one or more amino acid substitutions to decrease DNA binding activity, as defined above.

In one embodiment, the modified hyperactive PiggyBac transposase comprises at least one substitution to increase excision activity at position D450, and at least two substitutions to decrease DNA binding activity at positions N347, R372 and K375; numbering based on the wild-type hyperactive PiggyBac transposase with SEQ ID NO: 9; preferably said modified hyperactive PiggyBac transposase comprises the double mutations N347S and D450N or triple substitutions D450N, R372A and K375A; numbering based on the wild-type hyperactive PiggyBac transposase with SEQ ID NO: 9. In one embodiment, the modified transposase of hyperactive PiggyBac includes the double mutations N347S and D450N, said position number corresponding to the amino acid number of unmodified hyperactive Piggybac of SEQ ID NO: 9.

In some embodiments, the modified transposase of hyperactive PiggyBac does not comprise the triple mutations D450N, R372A and K375A, said position number corresponding to the amino acid number of unmodified hyperactive PiggyBac of SEQ ID NO: 9.

In one embodiment, the modified hyperactive PiggyBac transposase as disclosed in the previous embodiments further comprises at least one substitution in the region defined by the amino acid position numbers [158-169], for example A166S; and/or at least one substitution at position Y527, R518, K525 and/or N463; numbering based on the wild-type hyperactive PiggyBac transposase with SEQ ID NO: 9.

Typically, said modified hyperactive PiggyBac transposase comprises an amino acid sequence having at least 85%, at least 90%, at least 95% identity to the unmodified hyperactive PiggyBac transposase of SEQ ID NO: 1.

In one embodiment, said modified hyperactive PiggyBac transposase further comprises one or more of the following amino acid substitutions at positions selected from 34, 43, 117, 202, 230, 245, 268, 275, 277, 287, 290, 315, 325, 341, 346, 347, 350, 351, 356, 357, 388, 409, 412, 432, 447, 460, 461, 465, 517, 560, 564, 571, 573, 576, 586, 587, 589, 592, and 594; numbering based on the wild-type hyperactive PiggyBac transposase with SEQ ID NO: 9.

In one embodiment, said modified hyperactive PiggyBac transposase comprises one of the following amino acid substitutions or combinations of substitutions: V34M, T43I, Y177H, R202K, S230N, R245A, D268N, R275A, R277A, K287A, K290A, K287A/K290A, R315A, G325A, R341A, D346N, N347A, N347S, T350A, S351E, S351P, S351A, K356E, N357A, R388A, K409A, A411T, K412A, K432A, D447A, D447N, D450N, R460A, K461A, W465A, S517A, T560A, S564P, S571N, S573A, K576A, H586A, 1587A, M589V, S592G, F594L, D450N/R372A/K375A, R275A/R277A, K409A/K412A, R460A/K461A, R275A/R277A/N347S/K375A/T560A/S573A/M589V/S592G and R245A/R275A/R277A/R372A/W465A; numbering based on the wild-type hyperactive PiggyBac transposase with SEQ ID NO: 9.

In one embodiment, said modified hyperactive PiggyBac transposase comprises one of the following amino acid substitutions or combination of amino acid substitutions:
R372A/K375A/D450N,
R372A/K375A/R376A/D450N,
K375A/R376A/E377A/E380A/D450N,
R372A/K375A/R376A/E377A/E380A/D450N,
M194V,
M194V/R372A/K375A,
S351A/R372A/K375A/R388A/D450N/W465A/S573A/M589V/S592G/F594L,
R245A/R275A/R277A/R372A/W465A/M589V,
R275A/325A/R372A/T560A,
N347A/D450N,
N347S/D450N/T560A/S573A/F594L,
R202K/R275A/N347S/R372A/D450N/T560A/F594L,
R275A/N347S/K375A/D450N/S592G,
R275A/N347S/R372A/D450N/T560A/F594L,
R275A/R277A/N347S/R372A/D450N/T560A/S564P/F594L
R245A/N347S/R372A/D450N/T560A/S564P/S573A/S592G,
R277A/G325A/N347A/K375A/D450N/T560A/S564P/S573A/S592G/F594L,
V34M/R275A/G325A/N347S/S351A/R372A/K375A/D450N/T560A/S564P,
G325A/N347S/K375A/D450N/S573A/M589V/S592G,
S230N/R277A/N347S/K375A/D450N,
T43I/R372A/K375A/A411T/D450N,
G325A/N347S/S351A/K375A/D450N/S573A/M589V/S592G, or
Y177H/R275A/G325A/K375A/D450N/T560A/S564P/S592G;
   numbering based on the wild-type hyperactive PiggyBac transposase with SEQ ID NO: 9.

In one embodiment, said modified hyperactive PiggyBac transposase comprises one of the following amino acid substitutions or combination of amino acid substitutions:
K375A/R376A/E377A/E380A/D450N,
R372A/K375A/R376A/E377A/E380A/D450N,
M194V,
M194V/R372A/K375A,
R245A/R275A/R277A/R372A/W465A/M589V,
R275A/325A/R372A/T560A,
N347A/D450N,
N347S/D450N/T560A/S573A/F594L,
R202K/R275A/N347S/R372A/D450N/T560A/F594L,
R275A/N347S/K375A/D450N/S592G,
R275A/N347S/R372A/D450N/T560A/F594L,
R275A/R277A/N347S/R372A/D450N/T560A/S564P/F594L
R245A/N347S/R372A/D450N/T560A/S564P/S573A/S592G,
R277A/G325A/N347A/K375A/D450N/T560A/S564P/S573A/S592G/F594L,
G325A/N347S/K375A/D450N/S573A/M589V/S592G,
S230N/R277A/N347S/K375A/D450N,
G325A/N347S/S351A/K375A/D450N/S573A/M589V/S592G, or
Y177H/R275A/G325A/K375A/D450N/T560A/S564P/S592G;
   numbering based on the wild-type hyperactive PiggyBac transposase with SEQ ID NO: 9.

In a preferred embodiment, said modified hyperactive PiggyBac transposase comprises one of the following amino acid substitutions or combination of amino acid substitutions:
R372A/K375A/D450N,
S351A/R372A/K375A/R388A/D450N/W465A/S573A/M589V/S592G/F594L,
R245A/R275A/R277A/R372A/W465A/M589V,
N347A/D450N,
N347S/D450N/T560A/S573A/F594L,
R202K/R275A/N347S/R372A/D450N/T560A/F594L,
R275A/N347S/K375A/D450N/S592G,
R275A/N347S/R372A/D450N/T560A/F594L,
R275A/R277A/N347S/R372A/D450N/T560A/S564P/F594L,
R275A/325A/R372A/T560A,
R245A/N347S/R372A/D450N/T560A/S564P/S573A/S592G,
R277A/G325A/N347A/K375A/D450N/T560A/S564P/S573A/S592G/F594L,
G325A/N347S/K375A/D450N/S573A/M589V/S592G,
S230N/R277A/N347S/K375A/D450N,
G325A/N347S/S351A/K375A/D450N/S573A/M589V/S592G, or
Y177H/R275A/G325A/K375A/D450N/T560A/S564P/S592G;
   numbering based on the wild-type hyperactive PiggyBac transposase with SEQ ID NO: 9.

In one embodiment, said modified hyperactive PiggyBac transposase has an amino acid sequence selected among any of SEQ ID NO: 1-8, 10-18, 108-113, and 122-130.

In one embodiment, said modified hyperactive PiggyBac transposase has an amino acid sequence selected among any of SEQ ID NO: 1-8 and 10-18.

In one embodiment, said modified hyperactive PiggyBac transposase has an amino acid sequence selected among any of SEQ ID NO: 108-113.

In one embodiment, said modified hyperactive PiggyBac transposase has an amino acid sequence selected among any of SEQ ID NO: 122-130.

In one embodiment, the modified hyperactive PiggyBac transposase can comprise one or more substitutions relative to the wild-type hyperactive PiggyBac transposase that are involved in the conserved catalytic triad, e.g., at amino acid 268 and/or 346 (e.g., D268N and/or D346N); numbering based on the wild-type hyperactive PiggyBac transposase with SEQ ID NO: 9 or on the modified hyperactive PiggyBac transposase with SEQ ID NO: 11.

In one embodiment, the modified hyperactive PiggyBac transposase can comprise one or more substitutions relative to the wild-type hyperactive PiggyBac transposase that are critical for excision, e.g., at amino acid 287, 287/290 and/or 460/461 (e.g., K287A, K287A/K290A, and/or R460A/K461A); numbering based on the wild-type hyperactive PiggyBac transposase with SEQ ID NO: 9 or on the modified hyperactive PiggyBac transposase with SEQ ID NO: 12.

In one embodiment, the modified hyperactive PiggyBac transposase can comprise one or more substitutions relative to the wild-type hyperactive PiggyBac transposase that are involved in target joining, e.g., at amino acid 351, 356, and/or 379 (e.g., S351E, S351P, S351A, and/or K356E); numbering based on the wild-type hyperactive PiggyBac transposase with SEQ ID NO: 9 or on the modified hyperactive PiggyBac transposase with SEQ ID NO: 13.

In one embodiment, the modified hyperactive PiggyBac transposase can comprise one or more substitutions relative to the wild-type hyperactive PiggyBac transposase that are critical for integration, e.g., at amino acid 560, 564, 571, 573, 589, 592, and/or 594 (e.g., T560A, S564P, S571N, S573A, M589V, S592G, and/or F594L); numbering based on the wild-type hyperactive PiggyBac transposase with SEQ ID NO: 9 or on the modified hyperactive PiggyBac transposase with SEQ ID NO: 14.

In one embodiment, the modified hyperactive PiggyBac transposase can comprise one or more substitutions relative to hyPB that are involved in alignment, e.g., at amino acid 325, 347, 350, 357 and/or 465 (e.g., G325A, N347A, N347S, T350A and/or W465A); numbering based on the wild-type hyperactive PiggyBac transposase with SEQ ID NO: 9 or on the modified hyperactive PiggyBac transposase with SEQ ID NO: 15.

In one embodiment, the modified hyperactive PiggyBac transposase can comprise one or more substitutions relative to the wild-type hyperactive PiggyBac transposase that are well conserved, e.g., at amino acid 576 and/or 587 (e.g., K576A and/or I587A); numbering based on the wild-type hyperactive PiggyBac transposase with SEQ ID NO: 9 or on the modified hyperactive PiggyBac transposase with SEQ ID NO: 16.

In one embodiment, the modified hyperactive PiggyBac transposase can comprise one or more substitutions relative to the wild-type hyperactive PiggyBac transposase that are involved in Zn²⁺ binding, e.g., 586 (e.g., H586A); numbering based on the wild-type hyperactive PiggyBac transposase with SEQ ID NO: 9 or on the modified hyperactive PiggyBac transposase with SEQ ID NO: 17.

In one embodiment, the programmable transposase can comprise one or more substitutions relative to the wild-type hyperactive PiggyBac transposase that are involved in integration, e.g., 315, 341, 372, and/or 375 (e.g., R315A, R341A, R372A, and/or K375A); numbering based on the wild-type hyperactive PiggyBac transposase with SEQ ID NO: 9 or on the modified hyperactive PiggyBac transposase with SEQ ID NO: 18.

In one embodiment, the modified hyperactive PiggyBac transposase is selected for its high specificity of DNA integration into a genome compared to the wild-type hyperactive PiggyBac transposase. In one embodiment, the modified hyperactive PiggyBac transposase comprises an amino acid sequence having one or more of the modifications disclosed herein relative to any one of SEQ ID NOs: 9-18 and 108-113, and retains at least 80%, at least 85%, at least 90%, at least 95%, at least 96%, at least 97%, at least 98%, or at least 99% identical to the sequence set forth in SEQ ID NOs: 1-18 and 108-113, respectively.

As shown in the examples, newly developed hyperactive PiggyBac transposase substitutions library have been used to identify modified hyperactive PiggyBac which perform specific targeted transpositions. Modified hyperactive PiggyBac with positive targeted transposition were identified using such library.

In one embodiment, the modified hyperactive PiggyBac transposase can comprise a substitution of one or more of amino acids selected from amino acid: 245, 275, 275, 277, 325, 347, 351, 372, 375, 388, 450, 465, 560, 564, 573, 589, 592, 594; numbering based on the wild-type hyperactive PiggyBac transposase with SEQ ID NO: 9.

In one embodiment, the modified hyperactive PiggyBac transposase comprises one or more of the amino acid substitution selected from: R245A, R275A, R275A, R277A, R275A/R277A, G325A, N347A, N347S, S351E, S351P, S351A, R372A, K375A, R388A, D450N, W465A, T560A, S564P, S573A, M589V, S592G, or F594L; numbering based on the wild-type hyperactive PiggyBac transposase with SEQ ID NO: 9.

In one embodiment, the modified hyperactive PiggyBac transposase comprises the amino acid substitution D450N; numbering based on the wild-type hyperactive PiggyBac transposase with SEQ ID NO: 9.

In one embodiment, the modified hyperactive PiggyBac transposase comprises the amino acid modification N347A; numbering based on the wild-type hyperactive PiggyBac transposase with SEQ ID NO: 9. In another embodiment, the modified hyperactive PiggyBac transposase comprises the amino acid substitution N347S; numbering based on the wild-type hyperactive PiggyBac transposase with SEQ ID NO: 9.

In one embodiment, the modified hyperactive PiggyBac transposase comprises the double amino acid substitutions D450N and N347A; numbering based on the wild-type hyperactive PiggyBac transposase with SEQ ID NO: 9. In another embodiment, the modified hyperactive PiggyBac transposase comprises the double amino acid substitutions D450N and N347S; numbering based on the wild-type hyperactive PiggyBac transposase with SEQ ID NO: 9.

In one embodiment, the modified hyperactive PiggyBac transposase comprises the amino acid substitutions R372A, K375A and D450; numbering based on the wild-type hyperactive PiggyBac transposase with SEQ ID NO: 9.

In one embodiment, the modified hyperactive PiggyBac transposase comprises the amino acid substitutions R245A and D450; numbering based on the wild-type hyperactive PiggyBac transposase with SEQ ID NO: 9.

In one embodiment, the modified hyperactive PiggyBac transposase comprises the amino acid substitutions R245A, G325A, and S573P; numbering based on the wild-type hyperactive PiggyBac transposase with SEQ ID NO: 9.

In one embodiment, the modified hyperactive PiggyBac transposase comprises the amino acid modifications R245A, G325A, D450 and S573P; numbering based on the wild-type hyperactive PiggyBac transposase with SEQ ID NO: 9.

In one embodiment, the modified hyperactive PiggyBac transposase comprises the amino acid substitutions N347S and D450N; numbering based on the wild-type hyperactive PiggyBac transposase with SEQ ID NO: 9.

In one embodiment, the modified hyperactive PiggyBac transposase comprises the amino acid substitutions N347A and D450N; numbering based on the wild-type hyperactive PiggyBac transposase with SEQ ID NO: 9. In one embodiment, this modified hyperactive PiggyBac transposase comprises the amino acid sequence of SEQ ID NO: 110.

In one embodiment, the modified hyperactive PiggyBac transposase comprises one or several amino acid substitutions selected among L25F, R36A, I42K, G59D, I212K, N245S, K252A and/or Q271L; numbering based on the wild-type hyperactive PiggyBac transposase with SEQ ID NO: 9.

The modified hyperactive PiggyBac transposases provided herein can be fused to other elements disclosed herein, such as a site-specific DNA binding protein capable of binding and cleaving a target nucleic acid sequence; or can be used either alone, or in combination with such other elements. In one embodiment, the modified hyperactive PiggyBac transposases disclosed herein comprise the amino acid sequence of SEQ ID NO: 9, wherein:
amino acid at position 34 is V or M,
amino acid at position 43 is T or I,
amino acid at position 177 is Y or H,
amino acid at position 202 is R or K,
amino acid at position 230 is S or N,
amino acid at position 245 is A,
amino acid at position 268 is D or N,
amino acid at position 275 is R or A,
amino acid at position 277 is R or A,
amino acid at position 325 is A or G,
amino acid at position 347 is N, S or A,
amino acid at position 351 is E, P or A,
amino acid at position 372 is R or A,
amino acid at position 375 is A or K,
amino acid at position 388 is R or A,
amino acid at position 409 is K or A,
amino acid at position 411 is A or T,
amino acid at position 412 is K or A,
amino acid at position 450 is D or N,
amino acid at position 460 is R or A,
amino acid at position 465 is W or A,
amino acid at position 517 is S or A,
amino acid at position 560 is T or A,
amino acid at position 564 is P or S,
amino acid at position 571 is S or N,
amino acid at position 573 is S or A,
amino acid at position 576 is K or A,
amino acid at position 586 is H or A,
amino acid at position 587 is I or A,
amino acid at position 589 is M or V,
amino acid at position 592 is G or S, and/or
amino acid at position 594 is L or F.

In one embodiment, the transposase is a Sleeping Beauty transposase.

In one embodiment, the Sleeping Beauty transposase comprises or consists of an amino acid sequence having at least 85%, 90%, 95%, 96%, 97%, 98%, 99%, or 100% sequence identity to SEQ ID NO: 73. An exemplary nucleic acid sequence encoding this protein is as set forth in SEQ ID NO: 72.

In one embodiment, the transposase is a modified Sleeping Beauty transposase. In one embodiment, the modified Sleeping Beauty transposase comprises one or more substitutions as compared to the wild-type Sleeping Beauty transposase.

In one embodiment, the one or more substitutions in the Sleeping Beauty transposase are selected among L25F, R36A, I42K, G59D, I212K, N245S, K252A and/or Q271L; numbering based on the wild-type Sleeping Beauty transposase with SEQ ID NO: 73.

In one embodiment, the transposase is not a Himar1C9 mutant.

As will be further detailed below, certain aspects of the disclosure are also directed to vectors or plasmids (e.g., expression vectors, packaging vectors, etc.) comprising a nucleic acid construct encoding the transposase, in particular any of the hyperactive PiggyBac transposases or modified hyperactive PiggyBac transposases described herein; said vectors or plasmids being preferably suitable for expression in a host cell, e.g., mammalian cells, yeast cells, insect cells, plant cells, fungal cells, or algal cells.

In one embodiment, the first protein comprising or consisting of the site-specific DNA binding protein capable of binding and cleaving a target nucleic acid sequence (as described above), and the second protein comprising or consisting of a transposase (as described above), are fused together to form a fusion protein, either directly or indirectly via a linker.

Any embodiments relating to the site-specific DNA binding protein on one hand, and to the transposase on the other hand, apply mutatis mutandis in the case of the fusion protein described herein.

Hence, in one embodiment, the fusion protein comprises or consists of:
(i) a first protein comprising or consisting of an RNA-guided DNA nuclease, a zinc finger protein or a transcription activator like effector nuclease, as described above, and
(ii) a second protein comprising or consisting of a transposase, as described above.

In one embodiment, the fusion protein comprises or consists of:
(i) a first protein comprising or consisting of an RNA-guided DNA nuclease or zinc finger protein, as described above, and
(ii) a second protein comprising or consisting of a transposase, as described above.

In one embodiment, the fusion protein comprises or consists of:
(i) a first protein comprising or consisting of an RNA-guided DNA nuclease, as described above, and
(ii) a second protein comprising or consisting of a transposase, as described above.

In one embodiment, the fusion protein comprises or consists of:
(i) a first protein comprising or consisting of a Cas9 protein or a variant thereof, as described above, and
(ii) a second protein comprising or consisting of a hyperactive PiggyBac transposase or a modified hyperactive PiggyBac transposase, as described above; in particular a modified hyperactive PiggyBac transposase, as described above.

In one embodiment, the first protein and the second protein can be oriented in the fusion protein in either order.

In one embodiment, the fusion protein comprises or consists of the first protein fused at the C-terminal end of the second protein, either directly or indirectly via a linker. In other words, the fusion protein comprises or consists of, from N- to C-terminal: (i) the second protein (i.e., the transposase); (ii) optionally, a linker; and (iii) the first protein (i.e., the site-specific DNA binding protein, preferably the RNA-guided DNA nuclease; more preferably the Cas9 protein or variant thereof).

In one embodiment, the fusion protein comprises or consists of the first protein fused at the N-terminal end of the second protein, either directly or indirectly via a linker. In other words, the fusion protein comprises or consists of, from N- to C-terminal: (i) the first protein (i.e., the site-specific DNA binding protein, preferably the RNA-guided DNA nuclease; more preferably the Cas9 protein or variant thereof); (ii) optionally, a linker; and (iii) the second protein (i.e., the transposase).

In one embodiment, the fusion protein comprises a linker.

Suitable examples of linkers include peptidic linkers, between the first protein and the second protein (in any order).

In one embodiment, the peptidic linker is selected from the group comprising or consisting of (GGS)ₙ, (GGGGS)ₙ with SEQ ID NO: 114, (G)ₙ, (EAAAK)ₙ with SEQ ID NO: 115, XTEN linkers, and (XP)ₙ motif, and combinations of any of any of these, wherein n is independently an integer between 1 and 50.

In one embodiment, the linker is 12- to 24-amino acid long, or is encoded by a nucleic acid sequence that is 36- to 72-nucleotide long.

In one embodiment, the linker is a XTEN linker or a (GGS)ₙ linker.

In one embodiment, the linker is selected among the linkers shown in Table 1.

TABLE 1
Linkers

| | Nucleic acid sequence | Amino acid sequence |
|---|---|---|
| Linker | (SEQ ID NO) | (SEQ ID NO) |
| GGSx3 | ggtggatctggcggtggatctggtggcggt | GGSGGGSGGG |
| | (SEQ ID NO: 40) | (SEQ ID NO: 41) |
| GGS4x | ggagggagtggtgggtccggtggtagtggcggatcc | GGSGGSGGSGGS |
| | (SEQ ID NO: 42) | (SEQ ID NO: 43) |
| GGS5x | ggaggctccggtgggtctggtgggagcggtggtagt | GGSGGSGGSGGSGGS |
| | ggcggatcc | (SEQ ID NO: 45) |
| | (SEQ ID NO: 44) | |
| GGS6x | ggaggcagtggtgggagcggtggttccgggggtagt | GGSGGSGGSGGSGGSGGS |
| | ggtggttccgggggatcc | (SEQ ID NO: 47) |
| | (SEQ ID NO: 46) | |
| GGS7x | ggaggttctggaggctccggtgggtccgggggaagt | GGSGGSGGSGGSGGSGGS |
| | ggggggtcaggcggatcaggaggatcc | GGS |
| | (SEQ ID NO: 48) | (SEQ ID NO: 49) |
| GGS8x | ggaggtagcggaggttccggagggagcggcgggag | GGSGGSGGSGGSGGSGGS |
| | tgggggaagcgggggaagtggaggatccggggga | GGS |
| | ggatcc | (SEQ ID NO: 51) |
| | (SEQ ID NO: 50) | |
| Linker | tccggtagcgaaacaccggggacttcagaatcggcca | SGSETPGTSESATPES |
| XTEN | ccccggagtct | (SEQ ID NO: 53) |
| | (SEQ ID NO: 52) | |
| Linker B | ggaagcgccggtagtgcggctgggtctggcgagttc | GSAGSAAGSGEF |
| | (SEQ ID NO: 54) | (SEQ ID NO: 55) |

In one embodiment, the linker comprises an amino acid sequence selected from the group comprising or consisting of SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, or any combination thereof; respectively encoded by the exemplary nucleic acid sequence of SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54.

In one embodiment, the linker comprises or consists of the amino acid sequence of SEQ ID NO: 41; encoded by the exemplary nucleic acid sequence of SEQ ID NO: 40.

Also provided herein are fusion proteins obtained from the expression of any of the nucleic acid constructs provided in this disclosure.

In one embodiment, the fusion protein is a triple fusion protein.

Such triple fusion protein can comprise or consist of:
one first protein (i.e., one site-specific DNA binding protein) and two second protein (i.e., two transposases); or
two first protein (i.e., two site-specific DNA binding proteins) and one second protein (i.e., one transposase).

In one embodiment, the triple fusion comprises or consists of one first protein (i.e., one site-specific DNA binding protein) and two second protein (i.e., two transposases), and the triple fusion comprises from N- to C-terminal:
(i) the site-specific DNA binding protein, (ii) a first transposase; (iii) a second transposase; or
(i) a first transposase; (ii) the site-specific DNA binding protein, (iii) a second transposase; or
(i) a first transposase; (ii) a second transposase, (iii) the site-specific DNA binding protein.

In one embodiment, the first and second transposases are identical. In one embodiment, the first and second transposases are different. For example, the first transposase can be a hyperactive PiggyBac transposase and the second transposase can be a modified hyperactive PiggyBac transposase, chosen among any of the modified hyperactive PiggyBac transposases described herein. Alternatively, both the first and second transposases can be modified hyperactive PiggyBac transposases, but each bearing a different substitution or different combination of substitutions as described herein.

In one embodiment, the first and second transposases are capable of forming a functional dimer.

In one embodiment, the triple fusion comprises or consists of two first protein (i.e., two site-specific DNA binding proteins) and one second protein (i.e., one transposase), and the triple fusion comprises from N- to C-terminal:
(i) a first site-specific DNA binding protein, (ii) a second site-specific DNA binding protein; (iii) the transposase; or
(i) a first site-specific DNA binding protein; (ii) the transposase, (iii) a second site-specific DNA binding protein; or
(i) the transposase; (ii) a first site-specific DNA binding protein, (iii) a second site-specific DNA binding protein.

In one embodiment, the first and second site-specific DNA binding proteins are identical. In one embodiment, the first and second site-specific DNA binding proteins are different. For example, the first site-specific DNA binding protein can be a Cas9 protein and the second site-specific DNA binding protein can be a variant of a Cas9 protein, chosen among any of the Cas9 protein variants described herein. Alternatively, both the first and second site-specific DNA binding proteins can be Cas9 protein variants, but each being a different variant.

In one embodiment, the triple fusion protein optionally comprises a linker between two of its proteins or between the three proteins.

Also disclosed herein is a fusion protein comprising:
(i) the second protein comprising or consisting of a transposase; or a nucleic acid construct encoding said second protein, as described above, and
(ii) an RNA-binding protein capable of binding to at least one specific RNA sequence; or a nucleic acid construct encoding said RNA-binding protein.

In one embodiment, the fusion protein comprises a linker, as described above.

In one embodiment, the second protein comprises or consists of a transposase, said transposase being a hyperactive PiggyBac with SEQ ID NO: 9. In one embodiment, the second protein comprises or consists of a transposase, said transposase being a modified hyperactive PiggyBac comprising one or more amino acid mutations as compared to the hyperactive PiggyBac with SEQ ID NO: 9. In particular, the modified hyperactive PiggyBac can be any of those disclosed herein.

In one embodiment, the transposase/RNA-binding protein fusion can be further fused to the first protein comprising or consisting of the site-specific DNA binding protein, as described above.

In some embodiments, the RNA-binding protein is a MS2 bacteriophage coat protein (MCP) or a fragment thereof.

In some embodiments, the MCP has at least 75%, 80%, 85%, 90%, 95%, 96%, 97%, 98%, 99% or 100% identity with SEQ ID NO: 132 (encoded, e.g., by the nucleic acid sequence with SEQ ID NO: 131).

In some embodiments, the RNA-binding protein is capable of binding to at least one specific RNA sequence, said RNA sequence comprising a tetraloop. The term “tetraloop” is used interchangeably with the terms “stem loop” and “hairpin loop”.

In some embodiments, the at least one tetraloop is a MS2 RNA tetraloop-binding sequence.

In some embodiments, the tetraloop is comprised within a guide RNA (gRNA). In certain embodiments, the gRNA is in a complex with a Cas9 protein, as described above.

In some embodiments, the gRNA comprises at least one MS2 RNA tetraloop-binding sequence. In some embodiments, the gRNA comprises more than one MS2 RNA tetraloop-binding sequences.

In some embodiments, the gRNA comprising the at least one MS2 RNA tetraloop-binding sequence has at least 75%, 80%, 85%, 90%, 95%, 96%, 97%, 98%, 99% or 100% identity with SEQ ID NO: 134 (encoded, e.g., by the DNA sequence with SEQ ID NO: 133).

In some embodiments, the MCP in the fusion protein binds non-covalently to at least one MS2 RNA tetraloop-binding sequence comprised in a gRNA itself non-covalently bound to a Cas9 protein; in particular, the binding of the fusion protein to the Cas9/gRNA complex directs the excision activity of the modified hyperactive PiggyBac transposase towards the site specifically recognized by the Cas9/gRNA complex.

As will be further detailed below, certain aspects of the disclosure are also directed to vectors or plasmids (e.g., expression vectors, packaging vectors, etc.) comprising a nucleic acid construct encoding the fusion protein described herein; said vectors or plasmids being preferably suitable for expression in a host cell, e.g., mammalian cells, yeast cells, insect cells, plant cells, fungal cells, or algal cells.

According to the invention, the composition can comprise the first protein and/or the second protein (or the fusion protein comprising both), either as proteins, as described above; or as nucleic acid constructs encoding these proteins.

Hence, in one embodiment, the composition of the invention comprises or consists of:
a) a nucleic acid construct encoding the first protein described above, comprising or consisting of a site-specific DNA binding protein capable of binding and cleaving a target nucleic acid sequence described above;
b) a nucleic acid construct encoding a second protein described above, comprising or consisting of a transposase; and
c) a nucleic acid construct comprising or consisting of a transgene encoding the laminin-α2 protein, functional variant or fragment thereof.

In another embodiment, the composition of the invention comprises or consists of
a) a nucleic acid construct encoding the fusion protein described above, comprising or consisting of (i) a first protein comprising or consisting of a site-specific DNA binding protein capable of binding and cleaving a target nucleic acid sequence, and (ii) a second protein comprising or consisting of a transposase; and
b) a nucleic acid construct comprising or consisting of a transgene encoding the laminin-α2 protein, functional variant or fragment thereof.

In one embodiment, the nucleic acid construct encoding the fusion protein further comprises a nucleic acid sequence encoding a linker between the first and the second protein, as described above; or in the case of a triple fusion protein, between two of its proteins or between the three proteins.

According to the disclosure, the first and second proteins, or the fusion protein comprising or consisting of said first and second proteins, enable and/or promote site-specific insertion of a transgene into a genome, in particular of the transgene encoding the laminin-α2 protein, functional variant or fragment thereof.

Some embodiments are directed to a plasmid or a vector (such as, e.g., an expression vector) comprising either:
a nucleic acid construct encoding the first protein; or
a nucleic acid construct encoding the second protein; or
a nucleic acid construct encoding the first protein and a nucleic acid construct encoding the second protein; or a nucleic acid construct encoding the fusion protein or triple fusion protein.

In some embodiments, the plasmid is a packaging plasmid. In some embodiments, the plasmid further comprises a polynucleotide encoding capsid proteins, e.g., gag and pol. In some embodiments, the plasmid is combined with a second plasmid comprising a polynucleotide that encode proteins for a viral envelope (envelope plasmid); and a third plasmid comprising a nucleic acid construct comprising the LAMA2 transgene, wherein when the combination is introduced into a production cell line (e.g., eukaryotic cells, prokaryotic cells and/or cell lines), a virus particle comprising the nucleic acid constructs encoding the LAMA2 transgene and the nucleic acid construct encoding either of the first protein, second protein, both first and second proteins or fusion protein, is produced.

In some embodiments, the plasmid is combined with a second plasmid comprising comprising a polynucleotide encoding capsid proteins, e.g., gag and pol (a packaging plasmid, wherein the packaging plasmid lacks a functional integrase), a third plasmid comprising a polynucleotide that encodes proteins for a viral envelope (envelope plasmid) and a fourth plasmid comprising a nucleic acid construct comprising the LAMA2 transgene, wherein when the combination is introduced into a production cell line (e.g., eukaryotic and prokaryotic cells and/or cell lines), a virus particle comprising the nucleic acid constructs comprising the LAMA2 transgene and the nucleic acid construct encoding either of the first protein, second protein, both first and second proteins or the fusion protein, is produced.

In one embodiment, the first protein, second protein, both first and second proteins or fusion protein, and/or the LAMA2 transgene, are delivered to a cell using a lentivirus particle.

In one embodiment, the nucleic acid construct comprises a first polynucleotide sequence encoding the first protein comprising or consisting of site-specific DNA binding protein engineered to bind a target nucleic acid sequence, a second polynucleotide sequence encoding the second protein comprising or consisting of a transposase that enables insertion of LAMA2 transgene into the genome, and optionally, a third polynucleotide sequence comprising a nucleic acid sequence encoding a linker between the first and second polynucleotides. In some embodiments, the first protein is a zinc finger protein or a Cas9 protein or variant thereof, as described above; and/or the second protein is a hyperactive PiggyBac transposase or modified hyperactive PiggyBac transposase, as described above.

Examples of suitable linkers to produce a fusion protein have been described hereabove.

In some embodiments, a linker is not needed because the first protein is expressed from a separate plasmid from the second protein.

In one embodiment, instead of using a linker, the first and/or the second polynucleotide sequences comprise nucleic acids encoding the first and second protein, respectively, and further comprise additional nucleotides in at least one of their ends that make the function of linker.

In one embodiment, the nucleic acid construct is in DNA or RNA form.

Also provided herein, are vectors comprising any of the nucleic acid constructs provided in this disclosure. Particularly, the vectors are suitable for expression in mammalian cells, yeast cells, insect cells, plant cells, fungal cells, or algal cells. Also provided herein, are host cells comprising any of the nucleic acid constructs or vectors provided in this disclosure.

According to the invention, the composition further comprises a nucleic acid construct comprising or consisting of a transgene encoding the laminin-α2 protein, functional variant or fragment thereof, also called herein “LAMA2 transgene”.

In one embodiment, the LAMA2 transgene may be any nucleic acid sequence encoding a laminin-α2 protein, in particular a wild-type mammalian, preferably human, laminin-α2 protein, a functional variant or fragment thereof.

In one embodiment, the LAMA2 transgene can comprise either the LAMA2 wild-type gene (or a functional variant or fragment thereof), a LAMA2 cDNA (or a functional variant or fragment thereof) or a LAMA2 minigene (or a functional variant or fragment thereof).

In one embodiment, the LAMA2 transgene comprises a full-length LAMA2 gene.

Laminin-α2 chain protein is encoded by the LAMA2 gene (Gene ID: 3908, updated on Nov. 24, 2020), which is transcribed and translated into a 390-kDa protein. Laminin-α2 is a component of a heterotrimeric, cross-shaped molecule known as laminin-211 (or merosin) composed of three subunits (alpha, beta and gamma) which are bound to each other by disulfide bonds.

The coding sequences of a number of different mammalian laminin-α2 proteins are known in the art, including, without limitation, the laminin-α2 protein from human, pig, chimpanzee, dog, cow, mouse, rabbit or rat, and can be easily found in sequence databases. Alternatively, the coding sequence may be easily determined by the skilled person based on the polypeptide sequence.

In particular, the LAMA2 transgene can encode a human laminin subunit α2 isoform a precursor (NCBI reference: NP_000417, submitted on Oct. 22, 2020) or a laminin subunit α2 isoform b precursor (NCBI reference: NP_001073291.2, submitted on Oct. 24, 2020).

In one embodiment, the LAMA2 transgene encodes the human laminin subunit α2 isoform a precursor, preferably with SEQ ID NO: 74.

After translation, the laminin-α2 chain is typically cleaved into an N-terminal fragment and a C-terminal fragment that are non-covalently associated with each other.

In one embodiment, the LAMA2 transgene can encode different human mature isoforms of the laminin-α2 protein, preferably selected from the group comprising or consisting of laminin subunit α2 isoform X1 (NCBI reference: XP_005267038.1, with SEQ ID NO: 116), laminin subunit α2 isoform X2 (NCBI reference: XP_011534122.1, with SEQ ID NO: 117), laminin subunit α2 isoform X3 (NCBI reference: XP_005267039.1, with SEQ ID NO: 118), laminin subunit α2 isoform X4 (NCBI reference: XP_016866340.1, with SEQ ID NO: 119), laminin subunit α2 isoform X5 (NCBI reference: XP_01686634.1, with SEQ ID NO: 120) and laminin subunit α2 isoform X6 (NCBI reference: XP_005267038.1, with SEQ ID NO: 121).

As used herein, the terms “fragment” or “functional fragment”, when referring to the laminin-α2 protein or the nucleic acid coding therefor, refers to a protein, polypeptide or nucleic acid derived from the laminin-α2 protein as described above which still retains the activity of the full-length laminin-α2 protein but whose sequence is not 100% identical to the full-length protein, polypeptide or nucleic acid. A functional fragment can possess more, fewer, or the same number of residues as the corresponding native molecule, and/or can contain one or more amino acid or nucleotide substitutions. Preferably, the functional fragment refers to a mature form of the laminin-α2 protein which does not comprises a signal peptide at the N-terminal end of the protein. The skilled artisan can readily determine to which part of the laminin-α2 protein the signal peptide corresponds, e.g., based on information publicly available on the Uniprot database.

In one embodiment, the LAMA2 transgene comprises the sequence with SEQ ID NO: 75. In one embodiment, the LAMA2 transgene comprises a human LAMA2 minigene comprising a synthetic intron with SEQ ID NO: 82. In one embodiment, said synthetic intron is included between the nucleobases at position 3925 and 3926 of the coding sequence with SEQ ID NO: 75.

In one embodiment, the LAMA2 transgene may be any nucleic acid sequence encoding a functional laminin-α2 protein variant which retains the activity of a full-length laminin-α2 protein. In particular, laminin-α2 protein variants retain the ability to bind to laminin-211 β- and γ-subunits by disulfide bonds to form a functional cross-shaped laminin-211 or laminin-221. Laminins form independent networks and are associated with type IV collagen networks via entactin, fibronectin, and perlecan. They also bind to cell membranes through integrin receptors and other plasma membrane molecules, such as the dystroglycan glycoprotein complex and lutheran blood group glycoprotein. Through these interactions, laminins critically contribute to cell attachment and differentiation, cell shape and movement, maintenance of tissue phenotype, and promotion of tissue survival.

Preferably, as used herein, the terms “variant” or “functional variant”, with reference to the laminin-α2 protein, refer to a polypeptide having an amino acid or nucleotide sequence having at least 70, 75, 80, 85, 90, 95 or 99% sequence identity to the wild-type laminin-α2 protein sequence.

More preferably, the terms “variant” or “functional variant” refer to a polypeptide having an amino acid sequence that differs from a wild-type laminin-α2 protein sequence by less than 30, 25, 20, 15, 10 or 5 substitutions, insertions and/or deletions. In one embodiment, the variant differs from the wild-type laminin-α2 protein sequence by one or more conservative substitutions, preferably by less than 15, 10 or 5 conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine).

In one embodiment, the LAMA2 transgene therefore comprises a sequence having at least 70, 75, 80, 85, 90, 95 or 99% sequence identity to SEQ ID NO: 75.

In one embodiment, the LAMA2 transgene may comprise an optimized sequence encoding the laminin-α2 protein, variant or fragment thereof.

The term “optimized”, in the context of a nucleic acid sequence, refers to codon optimization and means that a codon that typically expresses a bias for human is changed to a synonymous codon (i.e., a codon that codes for the same amino acid residue) that does not express a bias for human. Thus, the change in codon does not result in any amino acid changes in the encoded protein.

According to the invention, the LAMA2 transgene is comprised in a nucleic acid construct.

In one embodiment, the nucleic acid construct comprises the LAMA2 transgene operably linked to one or more control sequences that direct the expression of said transgene in cells.

The promoter contains transcriptional control sequences that mediate the expression of the LAMA2 transgene upon introduction into a host cell. The promoter may be any polynucleotide that shows transcriptional activity in cells including mutant, truncated, and hybrid promoters. The promoter may be a constitutive or inducible promoter, preferably a constitutive promoter, and more preferably a strong constitutive promoter.

Examples of suitable promoters include, but are not limited to, CMV promoter, preferably with SEQ ID NO: 76, CAG promoter, preferably with SEQ ID NO: 77, EF1-α promoter, preferably with SEQ ID NO: 78, SV-40 promoter, preferably with SEQ ID NO: 79 and hybrid albumin enhancer-α1-antitrypsin (EalbAAT) liver specific promoter, preferably with SEQ ID NO: 80.

In an alternative embodiment, the LAMA2 transgene can also be inserted into the genome of a cell in such a manner that its expression is driven by an endogenous promoter at or near the integration site. In particular, the nucleic acid construct comprising the LAMA2 transgene can contain a splice acceptor to ensure gene expression when the transgene is integrating into the intron of an actively-expressed gene such as the endogenous LAMA2 or albumin gene, to have an endogenous control of expression.

Splice acceptor sites provide signals to target the sequences following the splice acceptor site to be expressed as (Padgett et al., 1988. Annu Rev Biochem. 55:1119-1150). A splice acceptor site is a nucleotide sequence that is generally involved in RNA splicing to remove intronic RNA sequences. The splice acceptor site is normally involved in the excision of introns, during which it is bound by an RNA-protein complex referred to as a spliceosome, cleaved, and then joined to a splice donor site that has already been cleaved. Splice acceptor sequences are well known in the art and can be readily obtained from genes at a position between the exon and intron where they mediate splicing. Alternately, splice acceptor sites may be chemically or enzymatically synthesized. Splice acceptor (SA) sites typically end in AG dinucleotides that are highly conserved. The remaining nucleotides of the sequence are primarily cytidines and/or thymidines. In one embodiment, the splice acceptor has a sequence with SEQ ID NO: 81.

The control sequence may also include appropriate transcription initiation, termination, and enhancer sequences; efficient RNA processing signals such as splicing and polyadenylation signals; sequences that stabilize cytoplasmic mRNA; sequences that enhance translation efficiency (i.e., Kozak consensus sequence); and/or sequences that enhance protein stability. A great number of expression control sequences, e.g., native, constitutive, inducible and/or tissue-specific, are known in the art and may be utilized to drive expression of the LAMA2 transgene. Typically, the LAMA2 transgene is operably linked to a transcriptional promoter and a transcription terminator. Poly(A) signals typically consist of a) a consensus sequence AAUAAA, which has been shown to be required for both 3′-end cleavage and polyadenylation of premessenger RNA (pre-mRNA), as well as to promote downstream transcriptional termination, and b) additional elements upstream and downstream of the AAUAAA sequence that control the efficiency of utilization of AAUAAA as a poly(A) signal. There is considerable variability in these motifs in mammalian genes.

In one embodiment, the polyadenylation signal sequence in the nucleic acid construct is a polyadenylation signal sequence of a mammalian gene or a viral gene. Suitable polyadenylation signals include, but are not limited to, a SV40 early polyadenylation signal, a SV40 late polyadenylation signal, a HSV thymidine kinase polyadenylation signal, a protamine gene polyadenylation signal, an adenovirus 5 EIb polyadenylation signal, a growth hormone polyadenylation signal, a PBGD polyadenylation signal, as well as in silico designed polyadenylation signals (synthetic) and the like. In one embodiment, the polyadenylation signal sequence is a poly(A) having a sequence selected from the group comprising or consisting of SEQ ID NO: 83, 84 and 85.

In one embodiment, the nucleic acid construct comprising the LAMA2 transgene also comprises an insulator element, to increase transgene expression levels, avoid silencing, and reduce expression variability.

Insulators are a complex class of cis-acting regulatory sequences that prevent the spread of heterochromatin and silencing of genes (barrier activity) and have enhancer-blocking activity. Insulators are typically 300 bp to 2000 bp in length. There are many examples of insulators, including the CTCF insulator, the gypsy insulator, and the β-globin locus.

In one embodiment, insulator element sequences flank both ends of the nucleic acid construct.

In one embodiment, the nucleic acid construct comprising the LAMA2 transgene comprises insulator elements with SEQ ID NOs: 86 and 87.

In one embodiment, the transposase disclosed herein recognizes inverted terminal repeat (ITR) elements that carry transposase-binding sites to catalyze excision and subsequent reintegration of the element between ITRs.

Thus, in a particular embodiment, said nucleic acid construct comprising LAMA2 transgene is flanked by inverted terminal repeats (ITR) that carry transposase binding sites, preferably 5′-ITR and 3′-ITR of SEQ ID NO: 88 and 89.

In one embodiment, an increase of the size between ITRs positively regulates the transfer of the LAMA2 transgene into genomic DNA.

In one embodiment, the size between ITRs is at least 105 bp. In one embodiment, the size between ITRs is at least 200 bp. In one embodiment, the size between ITRs is at least 300 bp.

As used herein, the term “at least 300 bp” means 300 bp, 400 bp, 500 bp, 600 bp, 700 bp, 800 bp, 900 bp, 1 kb, 2 kb or more.

In one embodiment, the nucleic acid construct comprising the LAMA2 transgene is contained in an expression vector.

Examples of appropriate vectors include, but are not limited to, recombinant integrating or non-integrating viral vectors and vectors derived from recombinant bacteriophage DNA, plasmid DNA or cosmid DNA. In one embodiment, said vector is a plasmid vector, a minicircle vector or a doggy bone DNA donor vector. Preferably, the vector is a recombinant integrating or non-integrating viral vector. Examples of recombinant viral vectors include, but not limited to, vectors derived from herpes virus, retroviruses, lentivirus, vaccinia viruses, adenoviruses, adeno-associated viruses or bovine papilloma virus.

In one embodiment said viral vector is a lentiviral or retroviral vector. The present disclosure thus also relates to viral particles comprising a nucleic acid construct comprising the LAMA2 transgene, or an expression vector comprising said nucleic acid construct, as described above. In particular, the nucleic acid construct or the expression vector may be packaged into a virus capsid to generate a “viral particle”, also named “viral vector particle”.

In one embodiment, the nucleic acid construct or the expression vector is packaged into a lentiviral or AAV-derived capsid, to generate a “lentiviral particle” or “AAV particle”.

According to the invention, the first protein comprises or consists of a site-specific DNA binding protein capable of binding and cleaving a target nucleic acid sequence.

As used herein, a “target sequence” or “target nucleic acid sequence” or “target site” is a sequence that defines a portion of a nucleic acid, e.g., in a genome, to which a binding molecule will bind, provided sufficient conditions for binding exist.

Said first protein can be engineered to bind to any sequence of choice within the genome of a cell, called “target nucleic acid sequence”. For example, the sequence 5′-GAATTC-3′ is a target site for the EcoRI restriction endonuclease.

In one embodiment, the target nucleic acid sequence is within a safe harbor locus in a cell's genome. In one embodiment, the target nucleic acid sequence is within the endogenous LAMA2 gene in a cell's genome. In particular, the first and/or second protein can be engineered to bind and/or to integrate the transgene encoding the laminin-α2 protein within a safe harbor locus.

A “safe harbor locus” refers to a region of a cell's genome, where the integrated material can be adequately expressed without perturbing endogenous gene structure or function. Safe harbor loci include, but are not limited to, AAVS1 (intron 1 of PPP1R12C), HPRT, HI 1, hRosa26, albumin and F-A region. The safe harbor loci may be exons or introns of ubiquitously expressed genes and/or genes with tissue specific expression (e.g., muscle). Safe harbor loci can be selected from the group consisting of: exon 1, intron 1 or exon 2 of PPP1R12C, exon 1, intron 1 or exon 2 of HPRT, and exon 1, intron 1 or exon 2 of hRosa26, intron 1 of the albumin gene. A safe harbor locus may also include a region of the genome devoid of endogenous genes and with open chromatin that allows for the expression of the inserted transgene without perturbing the genome structure or function.

In one embodiment, the first and/or second protein is engineered to bind and integrate the LAMA2 transgene into the endogenous LAMA2 gene within the genome of a cell, in particular into intron 1 of the endogenous LAMA2 gene.

In one embodiment, the first protein, optionally in combination with a guide RNA, bind a target nucleic acid sequence selected from the group consisting of any one of SEQ ID NOs: 90-97.

In one embodiment, the composition of the invention further comprises a guide RNA.

As used herein, a “guide RNA”, “gRNA” or “single guide RNA” refers to a nucleic acid that promotes the specific targeting or homing of a gRNA/Cas complex to a target nucleic acid.

In particular, gRNA refers to an RNA molecule that comprises a transactivating crRNA (tracrRNA) and a crRNA. Preferably, said guide RNA corresponds to a crRNA and tracrRNA which can be used separately or fused together. The complementary sequence pairing with the target sequence recruits Cas proteins to bind and cleave the DNA at the target sequence.

In one embodiment, the guide RNA is engineered to comprise a complementary sequence to a part of a target sequence, preferably selected from the group consisting of any one of SEQ ID NOs: 90-97.

As used herein, the term “complementary sequence” refers to the sequence of a polynucleotide (e.g., part of crRNA or tracRNA) that can hybridize to another part of polynucleotides under standard low stringent conditions. Preferably, the sequences are complementary to each other pursuant to the complementarity between two nucleic acid strands relying on Watson-Crick base pairing between the strands, i.e., the inherent base pairing between adenine and thymine (A-T) nucleotides and guanine and cytosine (G-C) nucleotides.

The present disclosure also relates to a method for integrating a LAMA2 transgene into a target nucleic acid sequence within the genome of a cell. Said method comprises the step of introducing the composition as described above into a cell such that the first and second proteins (either separately or as part of the fusion protein described above) cleave the target nucleic acid sequence and integrate the LAMA2 transgene within said target nucleic acid sequence.

Said method involves introducing in the cell: the first and second proteins (either separately or as part of the fusion protein described above) or one or several nucleic acid constructs encoding the same; a guide RNA; and the nucleic acid construct comprising or consisting of a transgene encoding the laminin-α2 protein, functional variant or fragment thereof.

These elements may be synthesized in situ in the cell as a result of the introduction of nucleic acid construct(s) or expression vector(s) encoding said elements, as described above, into the cell. Alternatively, said elements may be produced outside the cell and then be introduced therein.

In one embodiment, a nucleic acid construct, an expression vector or a viral particle comprising the LAMA2 transgene as described above is introduced into the cell.

In one embodiment, one or several polynucleotides encoding the first and second proteins (either separately or as part of the fusion protein described above) may be transfected in mRNA form, which is introduced directly into the cells, for example by electroporation or lipid nanoparticles.

In one embodiment, the guide RNA may also be produced outside the cell and then introduced therein, for example by electroporation or lipid nanoparticles.

In one embodiment, the first and second proteins (either separately or as part of the fusion protein described above) can be introduced into the cell alone or pre-complexed with a guide RNA.

In one embodiment, the guide RNA and/or the first and second proteins (either separately or as part of the fusion protein described above) are encoded by a nucleic acid construct or expression vector.

Products (proteins and nucleic acids) according to the invention may be delivered inside cells or subcellular compartments by liposomal delivery means, polymeric carriers, chemical carriers, lipoplexes, polyplexes, dendrimers, nanoparticles, emulsion, natural endocytosis or phagocytose pathway as non-limiting examples, as well as physical methods such as electroporation.

Said nucleic acid construct or expression vector can be introduced into cell by any methods known in the art and include, as non-limiting examples, stable transformation methods in which the nucleic acid construct or expression vector is integrated into the cell genome, transient transformation methods in which the nucleic acid construct or expression vector is not integrated into the genome of the cell and virus-mediated methods. For example, transient transformation methods include for example microinjection, electroporation or particle bombardment.

Nucleic acid molecule or nucleic acid construct or expression vector according to the invention may be transferred into cells using any known technique including, but being not limited to calcium phosphate transfection, DEAE-Dextran transfection, electroporation, microinjection, biolistic, viral infection or liposome-mediated transfection.

In one embodiment, the RNA, preferably guide RNA or mRNA encoding the first and second proteins (either separately or as part of the fusion protein described above) can be produced in vitro by, e.g., in vitro transcription. The RNA may then be introduced into the cells by electroporation (as described for example in Almåsbak et al., 2011. Cytotherapy. 13(5):629-640; Rabinovich et al., 2009. Hum Gene Ther. 20(1):51-61; and Beatty et al., 2013. Cancer Immunol Res. 2(2):112-20).

Alternatively, RNA may be introduced by other means such as by liposomes or cationic molecules etc.

In one embodiment, nucleic acid construct(s) or vector(s) introduced into a cell may be expressed episomally or may be integrated into the genome of the cell.

In one embodiment, polynucleotides, such as guide RNA and mRNA encoding the first and second proteins (either separately or as part of the fusion protein described above) and/or nucleic acid construct comprising or consisting of the LAMA2 transgene, may be delivered to a cell or a patient by a nanoparticle, preferably a lipid nanoparticle (LNP). Any lipid or combination of lipids that are known in the art can be used to produce an LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1.

Examples of neutral lipids are: DPSC, DPPC, POPC, DOPE, and SM. Examples of PEG-modified lipids are: PEG-DMG, PEG-CerC14, and PEG-CerC20.

In one embodiment, the method is an in vitro or ex vivo method. The in vitro method is performed on a culture of cells.

In another aspect, the present disclosure relates to an isolated engineered cell obtainable or obtained by the method described above. Said engineered cell comprises at least a transgene encoding laminin-α2 protein as described above.

In one embodiment, the isolated engineered cell comprises at least one transgene encoding laminin-α2, including any one or several of the control sequences that direct the expression of said transgene in cells, as described above.

In some embodiments, the isolated engineered cell comprises at least one transgene encoding laminin-α2, including flanking ITRs as described above.

In one embodiment, the inter-ITR size is at least 105 bp, at least 200 bp, at least 300 bp or more.

The engineered cell of the disclosure may be used for ex vivo gene therapy purposes. In such embodiments, the first and second proteins (either separately or as part of the fusion protein described above), the guide RNA and the LAMA2 transgene, or the nucleic acid construct(s), expression vector(s) or viral particle(s) as described above, are introduced into cells. Said isolated cells can be subsequently transplanted to the patient or subject. This implanting step may be accomplished using any method of implantation known in the art. For example, the genetically modified cells may be injected directly in the patient's blood or injected into the desired muscle, otherwise administered to the patient. Transplanted cells can have an autologous, allogenic or heterologous origin. In particular, said cells may be myocytes isolated from donor or patient skeletal muscle, smooth muscle or cardiac muscle or mesenchymental stem cells from bone marrow cells or peripheral blood. For clinical use, cell isolation will generally be carried out under Good Manufacturing Practices (GMP) conditions.

Suitable cells include, but not limited to, eukaryotic and prokaryotic cells and/or cell lines. Preferably, said cells are eukaryotic cells such as mammalian cells, these include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. A person skilled in the art will choose the more appropriate cells according to the patient or subject to be transplanted.

Said engineered cells may also be muscle cells. As used herein, the term “muscle” refers to cardiac muscle (i.e. heart) and skeletal muscle. As used herein, the term “muscle cells” refers to myocytes, myotubes, myoblasts, and/or satellite cells.

Said engineered cell may be a cell with self-renewal and pluripotency properties, such as stem cells or induced pluripotent stem cells. Suitable stem cells also include for example, embryonic stem cells, induced pluripotent stem cells (iPSCs), hematopoietic stem cells, neuronal stem cells and mesenchymal stem cells. Stem cells are preferably mesenchymal stem cells. Mesenchymal stem cells (MSCs) are capable of differentiating into at least one of an osteoblast, a chondrocyte, an adipocyte, or a myocyte and may be isolated from any type of tissue. Generally, MSCs will be isolated from bone marrow, adipose tissue, umbilical cord, or peripheral blood. Methods for obtaining thereof are well known to a person skilled in the art. Induced pluripotent stem cells (also known as iPS cells or iPSCs) are a type of pluripotent stem cell that can be generated directly from adult cells. Yamanaka et al. induced iPS cells by transferring the Oct3/4, Sox2, Klf4 and c-Myc genes into mouse and human fibroblasts, and forcing the cells to express the genes (WO 2007/069666). Thomson et al. subsequently produced human iPS cells using Nanog and Lin28 in place of Klf4 and c-Myc (WO 2008/118820). In certain embodiments, the cells are myoblasts. The myoblasts may be derived from stem cells, for example, iPSCs including from iPSCs derived from patients with muscular disorders such as muscular dystrophy.

The composition according to the present disclosure is preferably used in the form of a pharmaceutical composition comprising a therapeutically effective amount of products according to the present invention such as nucleic acid construct encoding said LAMA2 transgene, nucleic acid construct encoding the first and second proteins (either separately or as part of the fusion protein described above), polynucleotide or nucleic acid construct comprising guide RNA, expression vector or viral particle encoding said products.

In the context of the disclosure, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present disclosure, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with cells. The solution or suspension may comprise additives which are compatible with non-viral vectors, viral-vectors and nanoparticles and do not prevent components entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The composition according to the present disclosure is used for therapy, in particular for the treatment of LAMA2-related muscular dystrophy.

LAMA2-related muscular dystrophy is a disorder that causes weakness and wasting (atrophy) of muscles used for movement (skeletal muscles). This condition varies in severity, from a severe, early-onset type to a milder, late-onset form.

Early-onset LAMA2-related muscular dystrophy is apparent at birth or within the first few months of life. It is considered part of a class of muscle disorders called congenital muscular dystrophies and is sometimes called congenital muscular dystrophy type 1A. Affected infants may have severe muscle weakness, lack of muscle tone (hypotonia), little spontaneous movement, and joint deformities (contractures). Weakness of the muscles in the face and throat can result in feeding difficulties and an inability to grow and gain weight at the expected rate. Respiratory insufficiency, which occurs when muscles in the chest are weakened, causes a weak cry and breathing problems that can lead to frequent, potentially life-threatening lung infections.

As affected children grow, they often develop an abnormal, gradually worsening side-to-side curvature of the spine (scoliosis) and inward curvature of the back (lordosis). Children with early-onset LAMA2-related muscular dystrophy often do not develop the ability to walk. Difficulty with speech may result from weakness of the facial muscles and an enlarged tongue. Seizures occur in about a third of individuals with early-onset LAMA2-related muscular dystrophy; rarely, heart complications occur in this form of the disorder.

Symptoms of late-onset LAMA2-related muscular dystrophy become evident later in childhood or adulthood, and are similar to those of a group of muscle disorders classified as limb-girdle muscular dystrophies. In late-onset LAMA2-related muscular dystrophy, the muscles most affected are those closest to the body (proximal muscles), specifically the muscles of the shoulders, upper arms, pelvic area, and thighs. Children with late-onset LAMA2-related muscular dystrophy sometimes have delayed development of motor skills such as walking, but generally achieve the ability to walk without assistance. Over time, they may develop rigidity of the back, joint contractures, scoliosis, and breathing problems. However, most affected individuals retain the ability to walk and climb stairs.

The disclosure provides also a method for treating a LAMA2-related muscular dystrophy according to the present disclosure, comprising: administering to a patient a therapeutically effective amount of the composition, engineered cell or pharmaceutical composition as described above.

A “therapeutically effective amount” refers to an amount effective, at dosages and for periods of time necessary to achieve the desired therapeutic result, and prevent, delay or reverse at least one or more signs or symptoms of LAMA2-related muscular dystrophy such as weakness and atrophy of skeletal muscles. The therapeutically effective amount of the product of the disclosure, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

As used herein, the terms “patient” or “individual” denote a mammal. Preferably, a patient or individual according to the disclosure is a human.

In the context of the disclosure, the terms “treating” or “treatment”, as used herein, mean reversing, alleviating or inhibiting the progress of LAMA2-related muscular dystrophy or condition to which such term applies, or reversing, alleviating or inhibiting the progress of one or more symptoms of the disorder or condition to which such term applies, in particular reduction of weakness and atrophy of skeletal muscles and/or improve of skeletal muscle function.

The pharmaceutical composition of the present disclosure is generally administered according to known procedures, at dosages and for periods of time effective to induce a therapeutic effect in the patient.

The administration can be systemic or local. Systemic administration is preferably parenteral such as subcutaneous (SC), intramuscular (IM), intravascular such as intravenous (IV) or intraarterial; intraperitoneal (IP); intradermal (ID), interstitial or else. The administration may be for example by injection or perfusion. In some preferred embodiments, the administration is parenteral, preferably intravascular such as intravenous (IV) or intraarterial. The practice of the present disclosure will employ, unless otherwise indicated, conventional techniques, which are within the skill of the art. Such techniques are explained fully in the literature.

Also provided by the present disclosure are kits for practicing the disclosed methods, as described herein. The kit can contain the first and second proteins (either separately or as part of the fusion protein described above) or nucleic acid constructs encoding the same, and a nucleic acid construct comprising the LAMA2 transgene as described herein. In some aspects, the kit can contain the lentiviral particles containing the nucleic acid constructs encoding the first and second proteins (either separately or as part of the fusion protein described above) and a nucleic acid construct comprising or consisting of the LAMA2 transgene as described herein.

The subject kit can further include instructions for using the components of the kit to practice the subject methods. The instructions for practicing the subject methods are generally recorded on a suitable recording medium. For example, the instructions can be printed on a substrate, such as paper or plastic, etc. As such, the instructions can be present in the kit as a package insert, in the labeling of the container of the kit or components thereof (i.e., associated with the packaging or subpackaging), etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

The disclosure typically relates to a composition or kit, comprising
a first composition including
(i) the first protein as defined herein, or a nucleic acid encoding said first protein,
(ii) the second protein as defined herein, or a nucleic acid encoding said second protein, and
(iii) a nucleic acid construct comprising or consisting of the LAMA2 transgene.

The disclosure also relates to a composition or kit, comprising
a first composition including
(i) a fusion protein as defined herein, or a nucleic acid encoding said fusion protein,
(ii) a nucleic acid construct comprising or consisting of the LAMA2 transgene.

In one embodiment, the composition or kit comprises exogenous nucleic acid in a minicircle, a plasmid or a viral vector, in particular in non-integrating viral vector, for example or non-integrating lentiviral vector. In one embodiment, the composition or kit as disclosed herein is comprised in a nanoparticle.

In one embodiment, the nucleic acid construct encoding the first and/or second proteins (either separately or as part of the fusion protein described above) is in form of RNA, DNA or protein, and the polynucleotide sequence encoding the LAMA2 transgene is in form of RNA or DNA, depending on the method of delivery. Particularly, the polynucleotide sequence encoding the exogenous nucleic acid is in form of RNA.

In one embodiment, the composition or kit is viral-free and the packaging vector is a nanoparticle, e.g., a polymeric or lipidic nanoparticle. The packaging vector can also be a carrier which is bound to the elements of the composition. In some embodiments, the composition is contained in a viral vector, particularly a lentiviral particle.

In one embodiment, the composition or kit comprises (a) the nucleic acid construct encoding the first and second proteins, either separately or as part of the fusion protein described above (e.g., comprising Cas9 and a transposase) in form of RNA, (b) a guide RNA if needed (e.g., as separate lineal single strand RNA molecule), and (c) a polynucleotide comprising or consisting of the LAMA2 transgene for insertion in DNA form (e.g. in a vector), contained in in or bound to a packaging vector.

In one embodiment, the composition comprises (a) the first and second proteins (either separately or as part of the fusion protein described above) described herein (e.g., comprising Cas9 and a transposase) in form of protein, (b) a guide RNA if needed (e.g., as separate lineal single strand RNA molecule), wherein the fusion protein and the guide RNA form a ribonucleic protein complex (RNP), and (c) a polynucleotide comprising or consisting of the LAMA2 transgene for insertion in DNA form (e.g. in a vector), contained in in or bound to a packaging vector.

In one embodiment, the composition comprises (a) the nucleic acid construct encoding the first and second proteins, either separately or as part of the fusion protein described above (e.g., comprising Cas9 and a transposase) in form of DNA, (b) a guide RNA if needed (e.g., as separate lineal RNA molecule or as DNA in a vector), and (c) a polynucleotide comprising or consisting of the LAMA2 transgene for insertion in DNA form (e.g. in a vector), contained in in or bound to a packaging vector.

In one embodiment, the composition comprises (a) the first and second proteins, either separately or as part of the fusion protein described above (e.g., comprising Cas9 and an integrase) in form of protein, (b) a guide RNA if needed (e.g., as separate RNA molecule complexing with the fusion protein), and (c) a polynucleotide comprising or consisting of the LAMA2 transgene for insertion, contained in in or bound to a packaging vector. In a particular embodiment, the packaging vector is a lentiviral particle. In some embodiments, the first and second proteins, either separately or as part of the fusion protein described above is/are bound to the lentiviral capsid by means of gag-pol or VPR (Viral Protein R). In some embodiments, the polynucleotide comprising or consisting of the LAMA2 transgene is in form of RNA as payload of the integrase.

In one embodiment, when the first protein is a ZFP, the guide RNA can not be needed.

The disclosure typically relates to a kit, comprising:
a first composition including
   (i) the first and second proteins, either separately or as part of the fusion protein described above, or a nucleic acid encoding the same, and wherein said first and second proteins comprise an amino acid sequence of a first guided RNA nickase Cas9, typically SpCas9 nickase with SEQ ID NO: 65 and a modified hyperactive PiggyBac, and,
   (ii) a first guided RNA nucleic acid,
a second composition including
   (iii) the first and second proteins, either separately or as part of the fusion protein described above, or a nucleic acid encoding the same, and wherein said first and second proteins comprise an amino sequence of a second guided RNA nickase Cas9, typically SaCas9 nickase with SEQ ID NO: 66 and a modified hyperactive Piggybac,
   (iv) a second guided RNA nucleic acid,
and a nucleic acid construct comprising LAMA2 transgene;
wherein each of the first and second proteins (in each of the first and second composition) are capable of heterodimerization and making double cuts determined by the first and second guided RNA at adjacent sites of a genomic DNA region, and optionally inserting said nucleic acid between the adjacent sites.

Some additional embodiments are disclosed hereafter.

E1: a composition comprising: a) a fusion protein comprising a first protein consisting of a site-specific DNA binding protein capable of binding a target nucleic acid sequence, preferably comprised within intron 1 of albumin; intron 1 of Lama2 or Rosa 26 locus and a second protein consisting of a transposase; or a nucleic acid construct, preferably mRNA encoding said fusion protein, b) a nucleic acid construct comprising a transgene encoding laminin-α2 protein, functional variant or fragment thereof.

E2: the transgene encodes laminin-α2 protein of SEQ ID NO: 74.

E3: the nucleic acid construct of b) comprises a promoter selected from the group consisting of: CMV promoter of SEQ ID NO: 76, CAG promoter of SEQ ID NO: 77, EF1-alpha promoter of SEQ ID NO: 78, SV-40 promoter of SEQ ID NO: 79 and EalbAAT promoter of SEQ ID NO: 80 or a splice acceptor of SEQ ID NO: 81.

E4: the nucleic acid construct of b) comprises a poly(A) signal sequence, preferably selected from the group consisting of SEQ ID NO: 83-85.

E5: the nucleic acid construct of b) can also comprises an insulator element, preferably selected from the group consisting of SEQ ID NO: 86-87.

E6: the nucleic acid construct of b) is flanked by inverted terminal repeats (ITR), preferably by 5′-ITR and 3′-ITR of SEQ ID NO: 88 and 89.

E7: the nucleic acid construct of b) may be comprised in a vector selected from the group consisting of: plasmid vector, a minicircle vector, a doggy bone DNA donor vector, a lentivirus vector and retrovirus vector.

E8: the site-specific DNA binding protein is RNA-guided nuclease comprising a Cas protein and the composition further comprises a guide RNA including a complementary sequence to a target nucleic acid sequence for integrating said LAMA2 transgene in a specific site of a genome of cell, preferably said Cas protein is S. pyogenes Cas 9 protein.

E9: the guide RNA comprises a complementary sequence of sequences selected from the group consisting of: SEQ ID NO: 90-97.

E10: the transposase is a modified hyperactive Piggybac transposase or Sleepy Beauty transposase, preferably a modified hyperactive PiggyBac transposase comprising one or more amino acid mutations to increase excision activity as compared to unmodified hyperactive Piggybac, and one or more amino acid mutations to decrease DNA binding activity as compared to unmodified hyperactive Piggybac.

E11: the hyperactive PiggyBac transposase is a modified hyperactive PiggyBac transposase comprising at least one mutation of amino acid selected from the group consisting of: M194, R245, G325, R372, K375, R376, E377, E380, D450 and S573, preferably comprising mutations of amino acids D450, R372 and K375, said position number corresponding to the amino acid number of unmodified hyperactive Piggybac of SEQ ID NO: 9.

E12: the transposase is preferably fused N-terminally to said site-specific DNA binding protein by a linker, preferably a peptidic linker comprising GGS, XTEN or FOKI, more preferably XTEN of SEQ ID NO: 53.

E13: the composition is packaged within nanoparticle.

E14: an in vitro method for integrating LAMA2 transgene into a target nucleic acid sequence within the genome of a cell comprising introducing into a cell a composition as described above and an engineered cell obtainable by said method comprising the LAMA2 transgene integrated within its genome.

E15: a pharmaceutical composition comprising a composition or an engineered cell as described above, optionally in combination with one or more pharmaceutically acceptable excipients.

E16: the composition, engineered cell or pharmaceutical composition for use in therapy, in particular for use in the treatment of merosin-deficient congenital muscular dystrophy type 1A (MDC1A) in a subject in need thereof.

The invention will now be exemplified with the following examples, which are not limitative, with reference to the attached drawings.

### BRIEF DESCRIPTION OF THE DRAWINGS

FIG. 1: Representation of the different Lama2 payloads that have been produced for therapeutic gene replacement in Lama2 animal models of MCD1A.

FIG. 2: Viral copy number on infected Hek293T cells with GFP or Lama2 loaded lentivirus. Titer was calculated based on qPCR estimations of copy numbers. The increased cargo size due to Lama2 decreases viral production efficiency 2 orders of magnitude.

FIG. 3: FACS analysis of circulating white blood cells (A) and infiltrated muscle cells (B); after isolation from mice 4 weeks after transplantation. Comparison of empty lentivirus transduction and GFP loaded virus is shown.

FIG. 4: Experimental design and workflow for the ex vivo lentivirus mediated Lama2 gene transfer to bone marrow derived cells to treat MCD1A models.

FIG. 5: Relative strength of treated and untreated MCD1A models versus wild type animals 6 and 12 weeks after transplantation with Lama2 expressing bone marrow derived cells (A). Muscle strength gain after treatment in the same animals (B). In both cases results showed the analysis of grip strength assays.

FIG. 6: Kaplan Meier survival curves for treated and untreated MCD1A models versus wild type animals over times.

FIG. 7: Experimental design and workflow for the in vivo, viral free, transposase mediated Lama2 gene transfer into liver cells to treat MCD1A models.

FIG. 8: Transgene copy number (A; C) and transgene expression (B; D); measured by qPCR and RT-qPCR respectively 4 weeks after treatment in liver cells. Data of RFP gene reporter, stably integrated by hyperactive PiggyBac (A; B); and Lama2 gene stably integrated by a programmable transposase Cas9 fused to an engineered PiggyBac (C; D) are shown.

FIG. 9: Elisa test for presence of Laminin in circulating serum after 1 week and 4 weeks of treatment of MDCIA models with Lama2, co-transfected with or without insertion machinery for stable expression.

FIG. 10: (A) RFP transposon alone (episomal) or together with hyPB or FICAT R372A_K375A_D450N mRNA delivered with in vivo JetPEI reagent were used to target Rosa26 safe harbor in mouse genome. Relative copy number of RFP transgene in liver was measured by semiquantitative qPCR and normalized to relative double copies of Tfrc gene (diploid genomes). (B) Liver integration of minicircle luciferase transposon. Minicircle luciferase transposon, sgRNA targeting Rosa26 locus and FiCAT (Cas9-hyPB R372A_K375A_D450N) mRNA were delivered by hydrodynamic injection and luciferase signal was monitored. (C) Junction PCR between transposon 3′ ITR and Rosa26 locus in liver genomic DNA. Mice were injected hydrodynamically with FiCAT R372A_K375A_D450N plasmid DNA or mRNA, gRNA targeting Rosa26 locus and minicircle transposon GFP payload and sacrificed 5 weeks after injection. PCR was performed amplifying genomic+ strand integration. n=2-3 animals/condition, numbers correspond to different individuals. 66% of treated mice with FiCAT mRNA or pDNA shows targeted insertion. Size of the band detected in FICAT corresponds to the expected size of the amplified insertion. A higher size band is detected in the episomal sample considered background.

FIG. 11: (a) C2C12 cells were transduced with RFP transposon alone (episomal), or in combination with FiCAT R372A_K375A_D450N and gRNA targeting Lama2 gene (spacer 271.1), RFP positive cells were monitored for 2 weeks after transduction. Mean±SD of n=2 technical replicates plotted. Representative image of n=3. (b) Karyoplot showing detected insertions in the c2c12 genome. (c) Junction PCR between 3′ ITR and Lama2 locus is shown (down panel) in + strand (1, 3) and − strand (2, 4) payload insertion comparing FiCAT (3, 4) and episomal (1, 2) treated enriched populations. Representative image of n=3. (d) Coverage at the on-target junction (Lama2 site).

FIG. 12: Targeted insertion of ½ GFP transposon in the AAVS1 site in the reporter cell line, relative to the insertion with 300 bp inter-ITR size. Inter-ITR sizes of 105 bp, 200 bp and 300 bp were tested for the insertion of ½ GFP with Cas9 fused to either hyperactive PiggyBac (hyPB) R372A_K375A_D450N (Cas9-PBx3) or a dimer of hyPB R372A_K375A_D450N (Cas9-PBx3-PBx3).

FIG. 13. Programmable transposase can be engineered with different Cas variants, such as CasX, CjCas9 Cpf1 or SaCas9, some of them achieved similar results in terms of programmable insertion at the target site as with SpCas9. Each of the Cas variant tested were targeted to the specific target region of the split GFP reporter cell line with 3 independent gRNAs.

FIG. 14. Programmable insertion activity of FiCAT R372A-K375A-D450N using four different nuclease proteins. SpCas9 is used as control for programmable insertion with gRNA-TRAC-1 only (left). Each nuclease was used with three independent gRNAs (1-3) for targeted insertion in ½ GFP reporter cell line.

FIG. 15. (a) Editing activity by CasX (left) and Cpf1 (middle). (b) Editing activity by SaCas9 (left), CjCas9 (middle). Mean % of reads with indels +/−SD is shown for two technical repeats, representative image of N=3 biological replicates. SpCas9 targeting the TRAC-1 site was used for reference (right).

FIG. 16. Programmable transposase can be engineered as a dimer polypeptide of two hyPB domains and a Cas9 nuclease, resulting in better programmable insertion compared to Cas9-hyPB. Split GFP reporter cell line was used for the programmable insertion of split GFP transposon to the target site. The mutant of hyPB R372A-K375A-D450N has been used for the monomer or dimer fusion to Cas9. Conditions: 1-Negative control with only hyPB as insertion machinery; 2: Positive control of Cas9-hyPB R372A-K375A-D450N in pcDNA expression vector; 3: Positive control of Cas9-hyPB R372A-K375A-D450N in Lentivirus expression vector; 4: Cas9 nuclease fused to two units of hyPB R372A-K375A-D450N in C-terminal; 5: Cas9 nuclease fused to two units of hyPB R372A-K375A-D450N one in C-terminal and the other one in N-terminal.

FIG. 17. Several cycles of selection of cells where programmable transposition took place allowed for the selection of best mutant combinations from a library. We identified several mutants with better enrichment, and programmable insertion capacity than Cas9-hyPB R372A-K375A-D450N when fused to Cas9.

FIG. 18. On-target efficiency increases over cycles of selection. Bulk variants selected from each cycle were co-transfected with gRNA targeting AAVS1 and ½ GFP transposon into the reporter cell line. Quantity of plasmid was corrected by PB copy number to normalize for cloning efficiency.

FIG. 19. (A) On-target efficiencies of the top selected candidates. Six individual candidates were selected based on the highest on-target activity among 96 random clones selected from the last cycle. The individual on-target activities were compared to Cas9-hyPB R372A-K375A-D450N. (B) Logo showing the predominant PB residues in top on-target activity variants.

FIG. 20. Double stranded breaks, by Cas9 and a single gRNA (gRNA-TCR1 or AAVS1-3) or by nickase Cas9 and two gRNAs targeting at nearby positions (gRNA-TCR1 and AAVS1-3), and programmable DNA binding domain (ZnF) in fusion to modified hyPB (mutants R372A-K375A-D405N) results in targeted insertion. Co-localization of double stranded break and PB in the insertion site is required for efficient on-targeted insertion. This can be achieved by nuclease Cas9 or double cut by nickase Cas9.

FIG. 21. Programmable insertion activity of dimeric hyPB R372A-K375A-D450N fused with either SpCas9 or SaCas9 for targeted insertion in ½ GFP reporter cell line.

FIG. 22. Increase of on-target efficiency over cycles of selection. (A) Bulk variants selected from each cycle were co-transfected with gRNA targeting AAVS1 and ½ GFP transposon into the reporter cell line. Quantity of plasmid was corrected by PB copy number to normalize for cloning efficiency. (B) Lentiviruses expressing bulk variants of each cycle were produced and used to infect reporter the cell line.

FIG. 23. Specific target integration relative to FiCAT (hyPB R372A-K375A-D450N) of single mutants isolated from bulk variants after 4 and 5 cycles of cas9_PB library enrichment co transfected with gRNA tcr1 and ½ GFP MC transposon.

FIG. 24. Relative comparison of the programmable insertion activity for targeted insertion in ½ GFP reporter cell line. (A) Comparison between hyPB R372A-K375A-D450N fused with SpCas9 protein (left) and hyPB R372A-K375A-D450N fused with MCP protein with SpCas9 added separately (right). (B) Comparison between 3 hyPB mutants (R372A-K375A-D450N; R202K-R275A-N347S-R372A-D450N-T560A-F594L; and R275A-N347S-R372A-D450N-T560A-F594L) fused to MCP protein with SpCas9 added separately.

FIG. 25. Comparison of the programmable insertion activity for targeted insertion in ½ GFP reporter cell line. (A) Comparison between the co-expression of hyPB R372A-K375A-D450N and SpCas9 protein (left) and the fusion protein comprising hyPB R372A-K375A-D450N with SpCas9 protein (right). (B) Relative comparison between 3 hyPB mutants (R372A-K375A-D450N; R202K-R275A-N347S-R372A-D450N-T560A-F594L; and R275A-N347S-R372A-D450N-T560A-F594L) co-expressed with SpCas9.

FIG. 26. Relative comparison of the programmable insertion activity for targeted insertion in ½ GFP reporter cell line with the co-expression of a first fusion protein comprising SpCas and hyPB R372A-K375A-D450N, and a second fusion protein comprising MCP protein and hyPB mutants (R372A-K375A-D450N; R202K-R275A-N347S-R372A-D450N-T560A-F594L; and R275A-N347S-R372A-D450N-T560A-F594L).

FIG. 27. Relative comparison of the programmable insertion activity for targeted insertion in ½ GFP reporter cell line with the co-expression of a fusion protein comprising SpCas and hyPB R372A-K375A-D450N, and 3 hyPB mutants R372A-K375A-D450N; R202K-R275A-N347S-R372A-D450N-T560A-F594L; and R275A-N347S-R372A-D450N-T560A-F594L.

FIG. 28. Comparison of the programmable insertion activity for targeted insertion in ½ GFP reporter cell line between SpCas9 fused to a dimer of hyPB R272A-K275A-D450N (left) and SpCas9 fused to a first hyPB R272A-K275A-D450N and to a second hyPB mutant (right).

### EXAMPLES

### 1. LAMA2 Payload Architecture

A LAMA2-coding sequence including leader sequence for exportation, for either Mus musculus (SEQ ID NO: 94) or Homo sapiens (SEQ ID NO: 75), can be used. The LAMA2-coding sequence can contain sequence recoding of a part of the sequence for payload specific detection and tracking after gene transfer, to differentiate it from the endogenous gene present in the organism we are performing the gene editing.

This LAMA2 gene is cloned downstream of a constitutive promoter (SEQ ID NOs: 76-79), or tissue specific promoter (SEQ ID NO: 80), or splice acceptor (SEQ ID NO: 81). The version with the splice acceptor can be integrated on the first intron of an actively-expressed gene to have an endogenous control of expression.

This coding sequence can also have a synthetic intron (SEQ ID NO: 82) for a mini-gene version of the payload. The cassette of expression can contain poly-A signals (SEQ ID NOs: 83-85), and insulator elements (SEQ ID NOs: 86-87).

The payload can be in the form of:
1. A plasmid vector; containing bacterial origin of replication and antibiotic resistant 2. A minicircle vector, that in addition to the plasmid vector components also contains 32 SceI restriction enzyme target sites and 2 AttB and AttP recombination sites flanking the Lama2 expression cassette.
3. A doggy bone DNA donor vector, that in addition to the plasmid vector components also contains TelN target site flanking the Lama2 expression cassette, for linearization and protection of the cargo ends of the donor DNA.
4. A lentivirus or gamma retrovirus payload vector, with long terminal repeats flanking the Lama2 expression cassette in addition to lentivirus/retrovirus operational elements that could be engineered to reduce in size allowing for additional space for the therapeutic cargo.

In all these cases the Lama2 expression cassette can be flanked by inverted terminal repeats (ITRs) for transposase recognition (Piggybac and Sleeping Beauty).

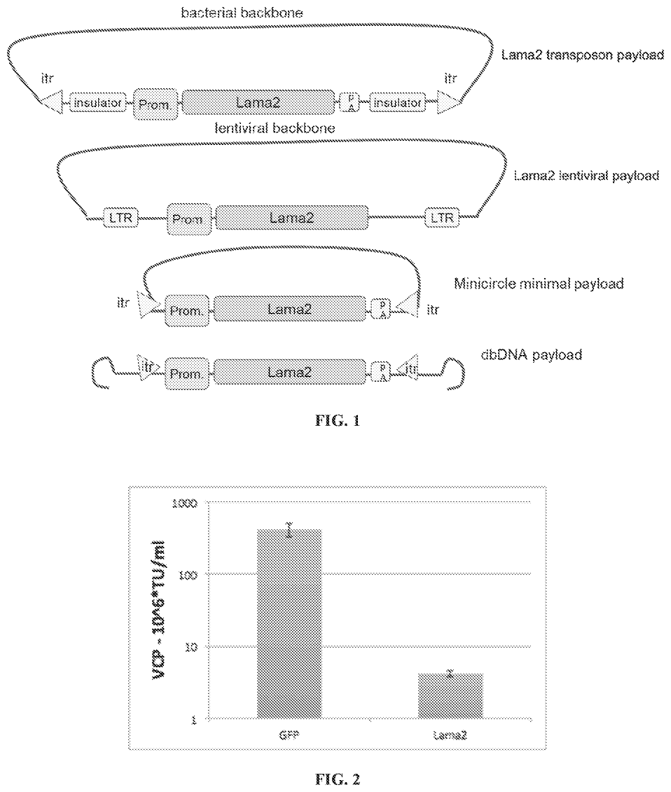

FIG. 1 shows a representation of the different cargos containing Lama2 transgene.

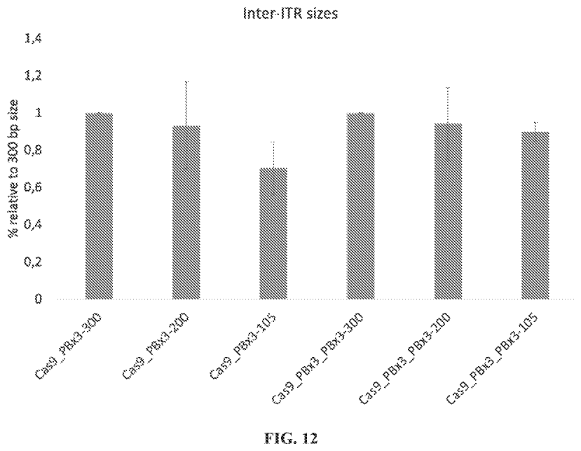

The inventors further tested if the size between ITRs of the payload affects the binding capacity of the fusion protein, and therefore its consequent transfer into the genome. Three inter-ITR sizes were tried for targeted insertion of the payload efficiency: 300 bp, 200 bp and 105 bp approximately in a minicircle (MC) payload of ½ GFP transposon with FiCAT (Cas9+hyperactive PiggyBac transposase mutated on residues R372A_K375A_D450N) and FiCAT dimer (Cas9+dimer of hyperactive PiggyBac transposase mutated on residues R372A_K375A_D450N) and gRNA targeting AAVS1 site in the reporter cell line (FIG. 12). Best inter-ITR size was 300 bp and 200 bp, while the targeted integration efficiency of 105 bp was lower.

In addition to lentivirus and retrovirus-based delivery, the different Lama2 payloads can be delivered using polyplex and lipid nanoparticles or transduced by electroporation or microinjection as naked nucleic acids into targeted cells.

### 2. Ex Vivo Transduction of Lama2 Payload

Lentiviral particles containing the Lama2 coding sequence of Mus musculus, under the control of CMV promoter were produced using standard protocols for second generation lentivirus production. Given the large size of this payload, the particles were concentrated, and viral titer was calculated by viral copy number qPCR estimation after infecting Hek293T cells (FIG. 2).

Although efficiency of production was twice as low compared to reporter gene GFP, this titer was sufficient for ex vivo transduction of cells that can be then used for cell therapy in Lama2 mice models. Two different approaches were taken. Isolated muscle stem cells were transduced and transplanted into muscle of recipient models. Alternatively, hematopoietic progenitors derived from bone marrow cells were transduced with the lentivirus containing Lama2 expression cassette and these progenitors can then be transplanted into conditioned models via intravenous injection.

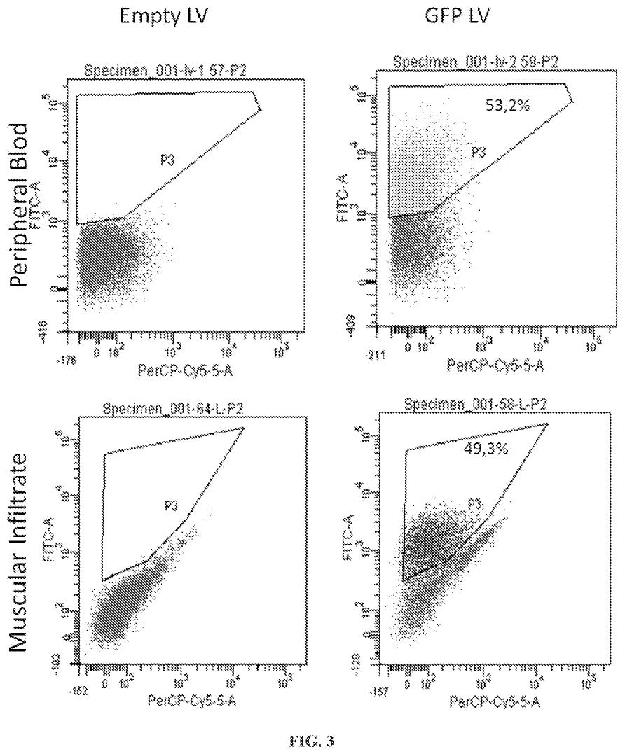

As a proof of concept for ex vivo treatment, the inventors isolated bone marrow derived hematopoietic progenitors and transduced them first with GFP expressing lentivirus, before transplanting them into conditioned mice. The inventors then check for engraftment 4 weeks after treatment by looking into GFP expression of white blood cells in circulating blood, and positive results were found (FIG. 3A). In addition, infiltration of circulating cells into the inflamed muscle was also checked and positive results were found (FIG. 3B).

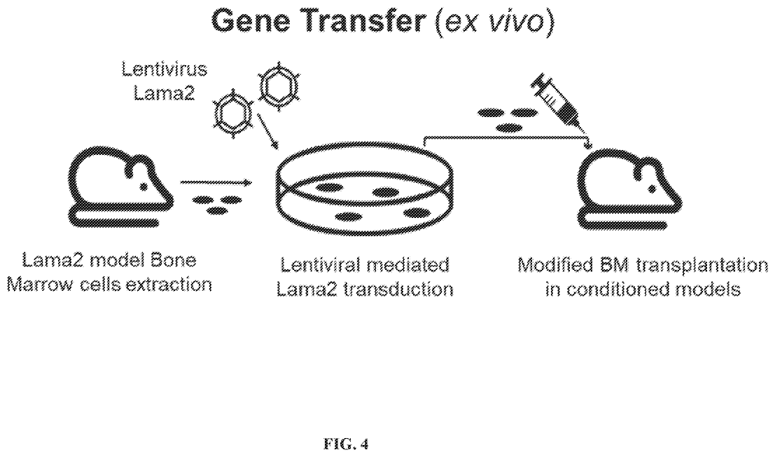

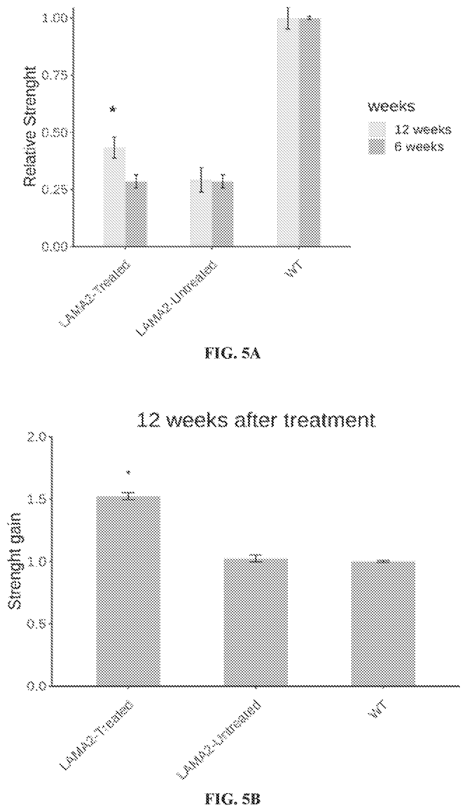

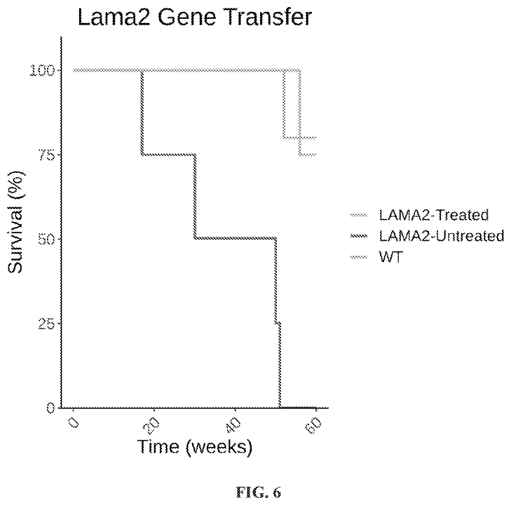

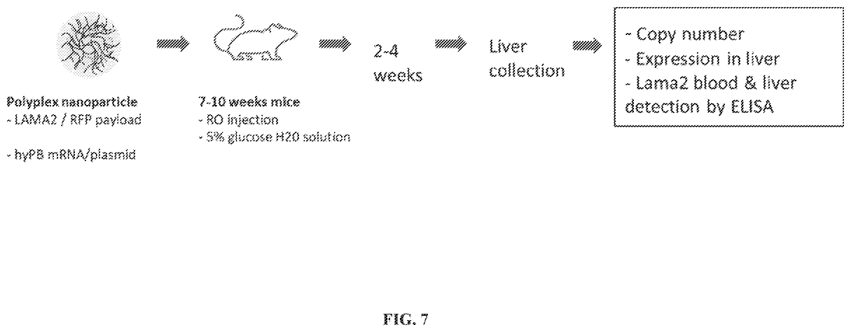

Given the positive infiltration and payload expression of ex vivo transduced hematopoietic progenitors, the inventors then repeated this experiment with Lama2 expressing lentiviral particles (FIG. 4). Mice recovered muscle strength 12 weeks after treatment measured by grip assay (FIGS. 5A and B). In addition, survival of the treated Lama2 animal models was also observed (FIG. 6), indicating the therapeutic value of ex vivo cell transduction and transplantation to treat MDC1A patients.

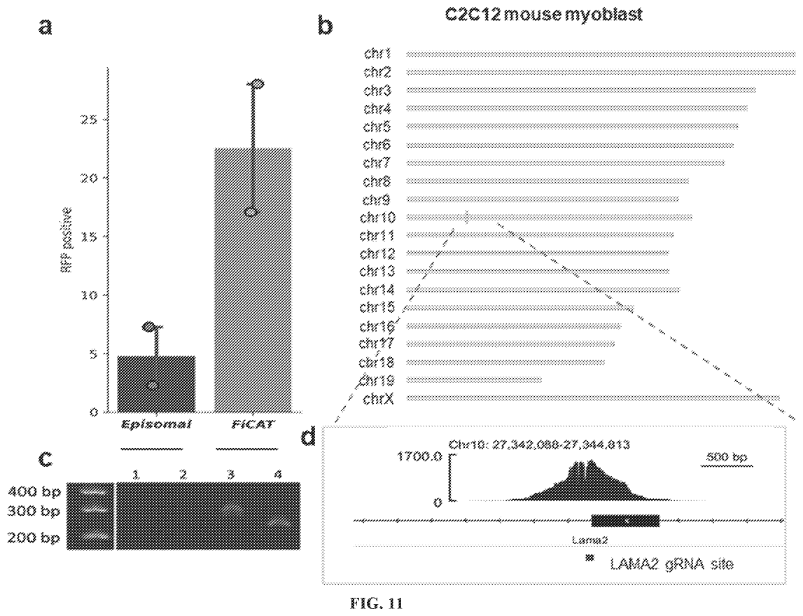

In another line of experiments, the Inventors performed LAMA2-targeting insertion of RFP reporter into the C2C12 murine myoblast cell line with an efficiency of ˜20% (FIG. 11). Junction PCR and (STAT)-PCR were used to measure on-target and off-target efficiency. This improved efficiency was the result of the use of a fusion protein comprising a Cas9 and a hyperactive PiggyBac transposase mutated on residues R372A_K375A_D450N, referred to as FiCAT.

### 3. In Vivo Transduction of Lama2 Payload

Given the capacity of the liver to function as a bioreactor for protein production in vivo; the inventors explored the possibility to transduce in vivo liver cells of animal models with the Lama2 expression vector using a viral free gene delivery approach based on transposase.

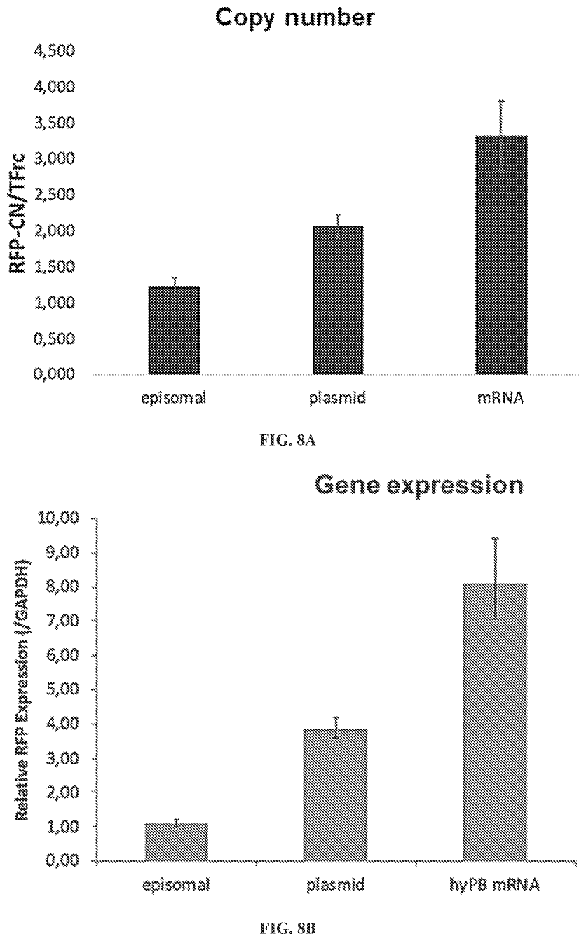

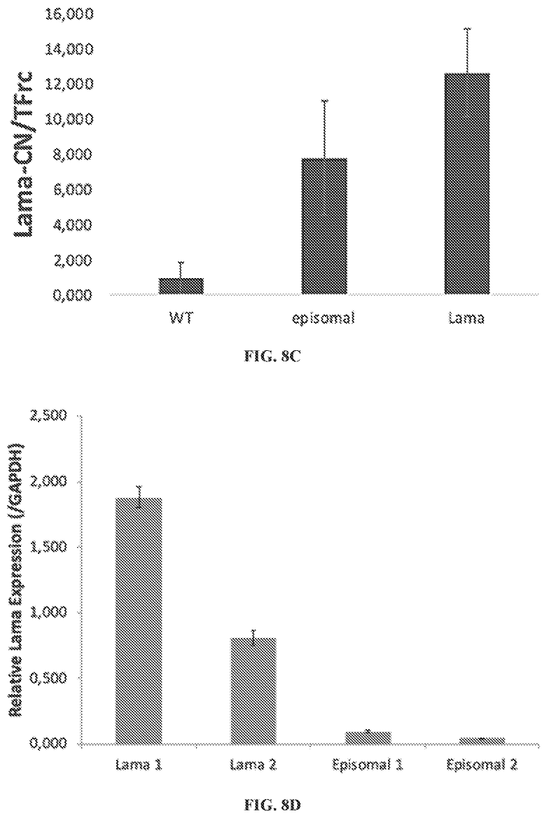

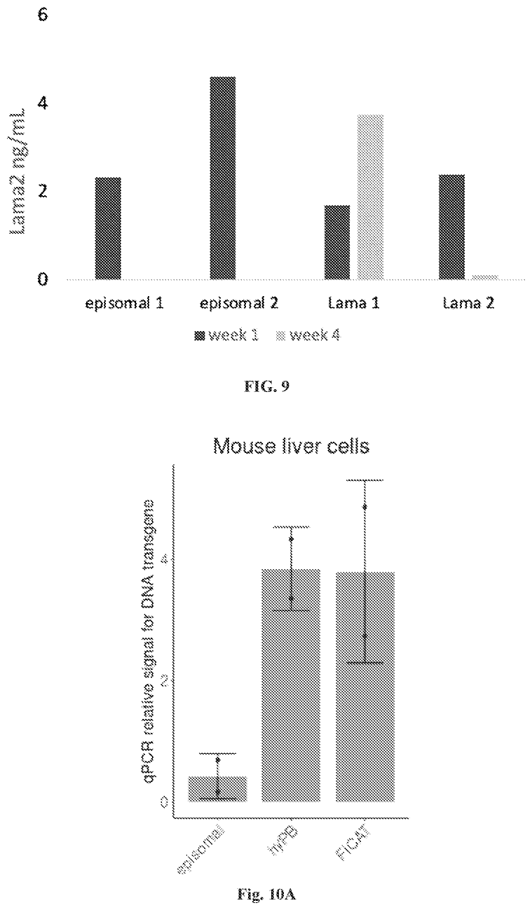

Hyperactive PiggyBac transposase or an engineered hyperactive Piggybac fusion transposase with a programmable nuclease protein (SpCas9) either as a plasmid vector or mRNA molecule and a transposon Lama2 plasmid vector formulated using In vivo JetPei (PolyPlus) (FIG. 7) were injected intravenously in animal models, and tissues were harvested after 3 to 4 weeks for payload copy number in liver (RFP data as proof of concept) (FIG. 8A, C), and expression analysis in liver (RFP, FIG. 8C; Lama2, FIG. 8D) and circulating blood for Lama2 (FIG. 9). 100 μL of blood was collected 1 week after injection and whole blood was collected at the end-point.

Transgene copy number in liver cells after 4 weeks of treatment showed positive results and was further compared to RFP reporter gene, confirming similar transduction efficiencies with different size payloads (FIG. 8A, B). Reference to episomal delivery was done when no transposase was co-delivered. Expression of transgene for the RFP reporter and Lama2 gene were also confirmed in liver cells (FIG. 8B, D), and Lama2 expression was detected on blood 1 week and 4 weeks after delivery (FIG. 9).

### 4. Improved Tools for In Vivo Gene Delivery

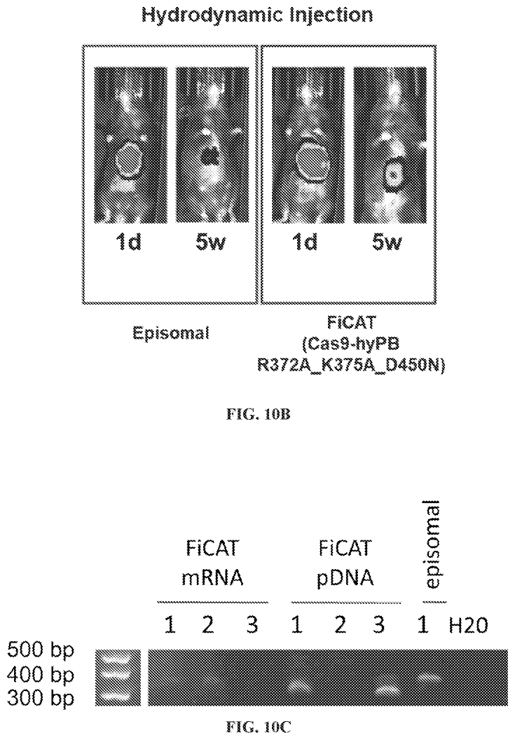

With the aim of improving the programmable transposase used for gene delivery in vivo, the inventors used both plasmid DNA (pDNA) and mRNA for FiCAT (Cas9-hyPB R372A_K375A_D450N) and delivered it to mice liver targeting Rosa26 genomic safe harbor (RFP or luciferase encoding transposon for proof of concept) either in plasmid or MC form. High copy number of transgene was observed compared to an endogenous gene TFRC (FIG. 10A) and maintained transgene expression overtime (FIG. 10B). PCR of the junction between 3′ ITR and genomic locus was used to measure the newly formed on-target insertion (FIG. 10C).

4.1. Results of Cas Variants Fused to hyPB

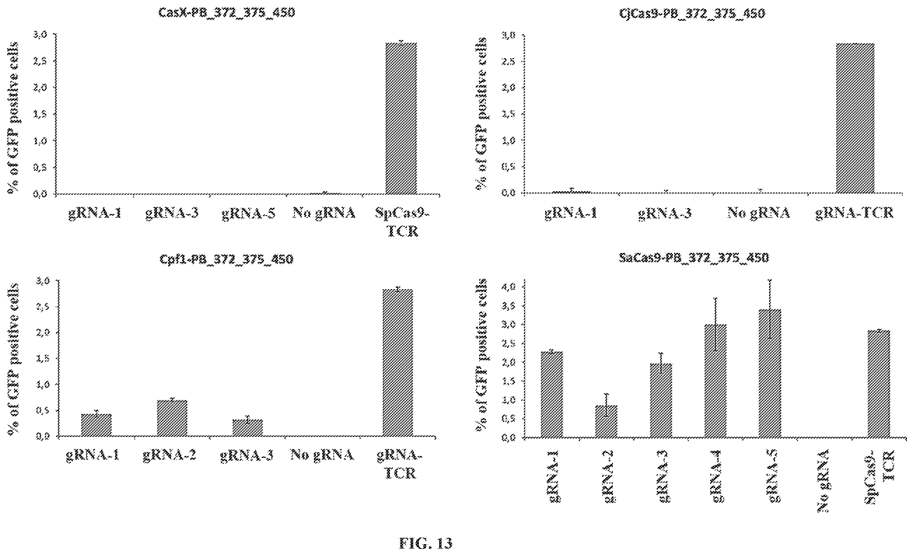

To further characterize the capacity of engineered hyPB to perform programmable transposition, we substituted the SpCas9 mudile of the programmable transposase tested, which other Cas proteins form different organisms with nuclease activity (namely SaCas9 of SEQ ID NO:72, cpf1 of SEQ ID NO:74, CasX of SEQ ID NO:75, and CjCas9 of SEQ ID NO: 29). Specific gRNA targeting the region upstream of the split GFP reporter were designed and cloned for Hershey cell line transfection. Targeted transposition was measured by means of GFP expression (FIG. 13).

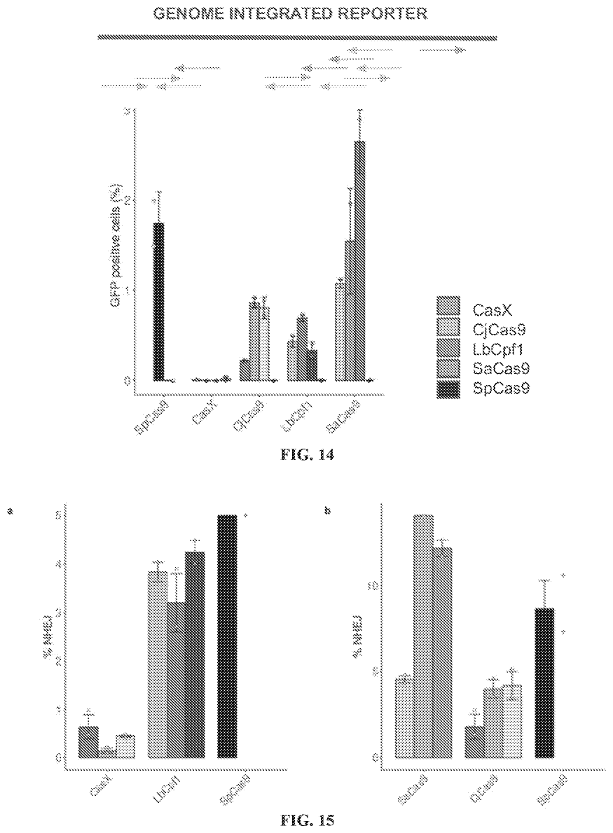

These results were confirmed in another line of experiments: we obtained good programmable insertion activity for CjCas9 and LbCpf1, while CasX did not achieve any programmable integration in our assay. Notably, SaCas9 had the highest levels of programmable insertion among the Cas proteins tested, with similar levels to SpCas9 fused to modified hyPB (FIG. 14). Indels were determined for the different Cas proteins used and the three different gRNA designed for each protein by Ilumina NGS (FIG. 15), shown for normalization purpose.

These positive results validate the engineered hyPB for programmable transposition to be useful for any sequence specific nuclease module.

### 4.2. Additional Results of Cas9 Fused to a Dimer of PB Mutants

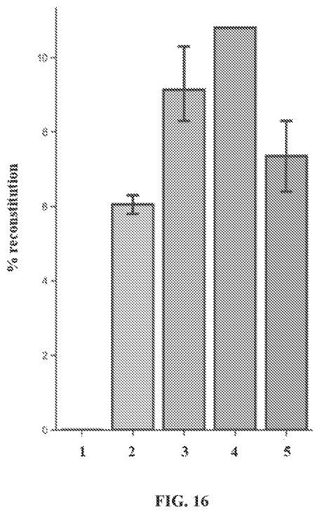

Given the nature of PB acting as dimers when performing transposition, we attempted to generate a fusion protein of Cas9 and hyPB R372A-K375A-D450N mutant. We compared the on-target activity of these fusion to Cas9-PB mutants alone. We observed a better performance of the configuration Cas9-PB-PB; while the configuration PB-Cas9-PB did not outperform the Cas9-PB monomeric fusion (FIG. 16). For the dimeric fusion to Cas9 we used a recorded version of hyPB R372A-K375A-D450N mutant to facilitate cloning and expression.

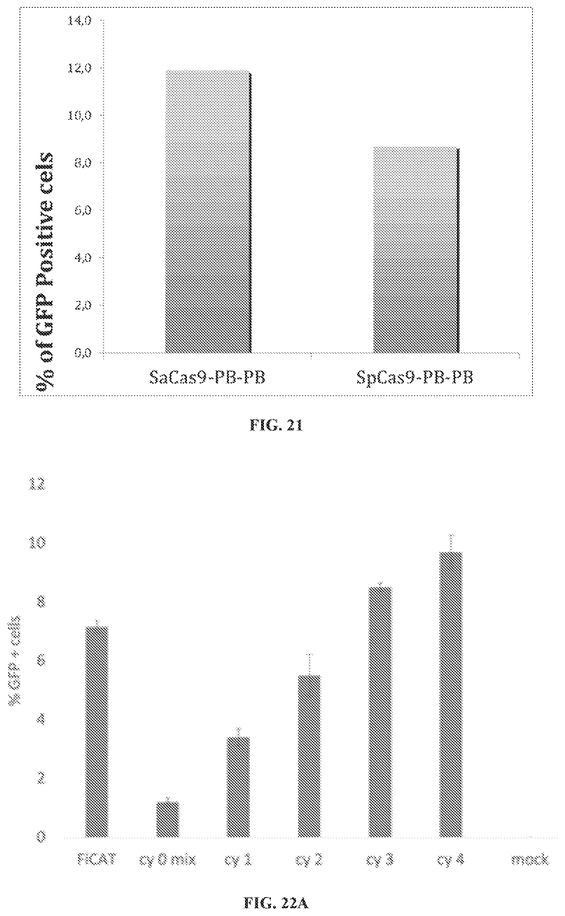

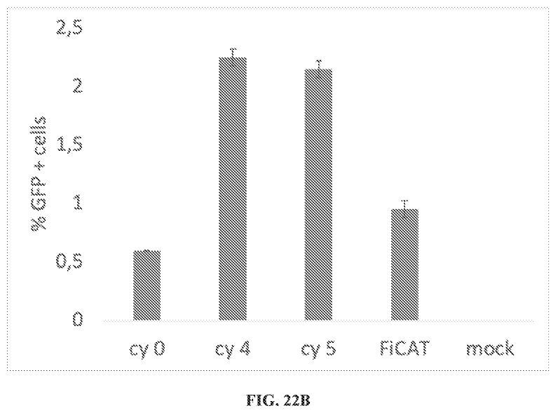

Interestingly, if the Cas9 fused to the dimeric hyPB R372A-K375A-D450N is the SaCas9 instead of SpCas9, the activity is further increased (FIG. 21). The increased performance of SaCas9 over SpCas9 with the dimeric hyPB is consistent with the results obtained with monomeric hyPB (FIGS. 13 and 14).

### 4.3. Results of ZNF-PB Mutants Rescue

We wanted to further explore the role of the double-strand break (DSB) activity induced by close by (4 nucleotides) target sites of two gRNAs promoting single stranded cuts by SpCas9 nickase variant (D10A) in facilitating targeted integration, while lowering off target activity by means of non-inducing DSB in off target sites. We used a Zinc finger-PB fusion for directed localization of the transposon, by fusion with D450N mutant and R372A-K375A-D450N mutant and complemented it with two on-site single stranded brakes by an independent nickase Cas9, or single DSB by Cas9 nuclease.

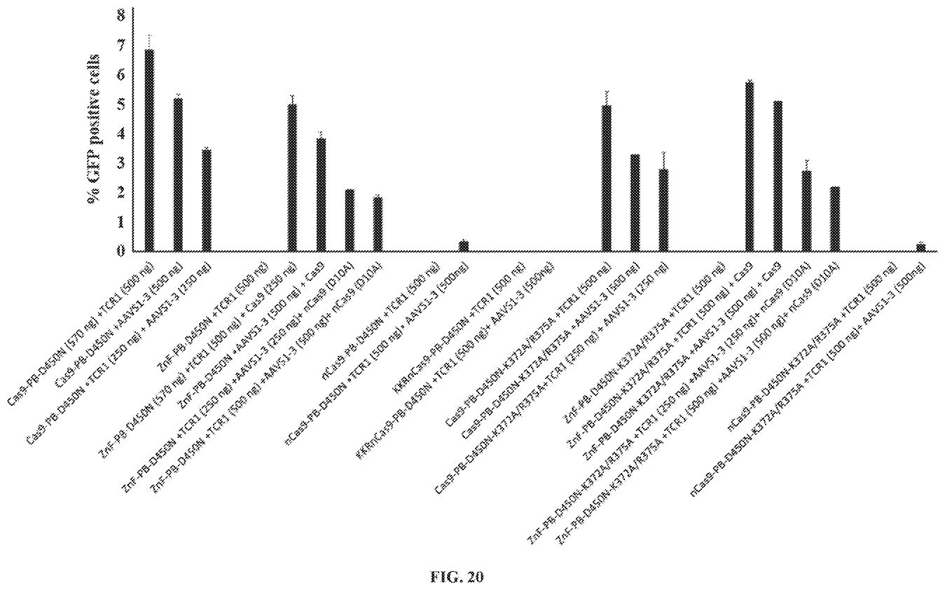

Znf-PB fusion exhibited no or very low targeted insertion activity that was rescued when combined with introducing DSBs near the Znf binding site with a single or dual gRNA guided-cas9 either nuclease or nickase (FIG. 20).

### 4.4. Results of Cas9-hyPB Mutant Variants

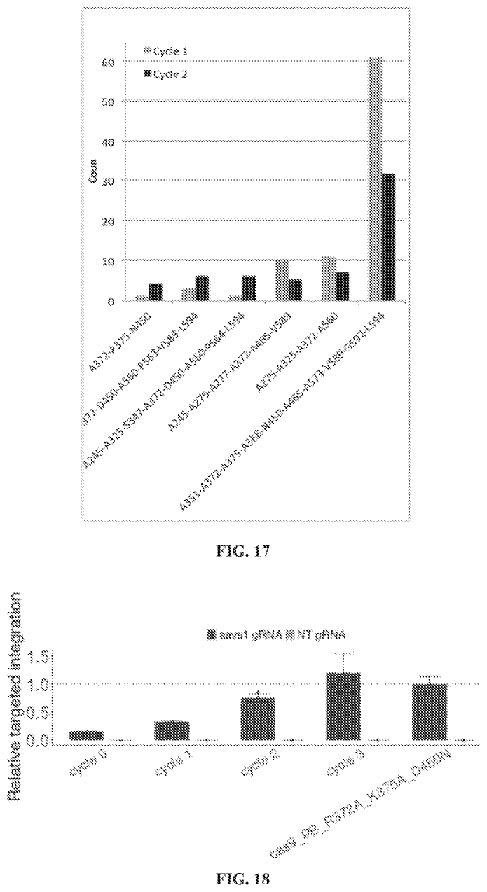

To further explore mutant combinations that could do programmable transposition with better efficiencies, several cycles of selection of cells were performed where GFP was reconstituted by programmable insertion of the split GFP reporter system. Interestingly we observed several combinations that out-performed the Cas9-hyPB R372A-K375A-D450N (FIG. 17). Especially worth of mention is variant of hyPB fused to Cas9 that are mutated on hyPB at AA: A351-A372-A375-A388-N450-A465-A573-V589-G592-L594 (also identified as SEQ ID NO:2), several fold enrich in the positive cells population compared to R372A-K375A-D450N (SEQ ID NO:1); and also A245-A275-A277-A372-A465-V589 (SEQ ID NO:3) and A275-A325-A372-A560 (SEQ ID NO:4) to a lesser extent.

In another line of experiments, PiggyBac DNA library was produced by Twist Bioscience, cloned in fusion with cas9 into a lentiviral vector and transformed into stb4 competent cells, ensuring ×100 variant complexity. Plasmids were purified by maxiprep and cotransfected with lentivirus packaging plasmids into Hek293T cells. Lentivirus was used to infect ½ GFP reporter cell line. Infected cells were transfected with the ½ GFP transposon and gRNA targeting AAVS1 sequence. GFP positive cells were selected by flow cytometry sorting and genomic DNA was extracted. PB was amplified from the extracted gDNA, recloned into lentiviral vector to restart a new cycle. Best performing programmable transposase variants were selected and transfected individually with AAVS1 gRNA and MC ½ GFP.

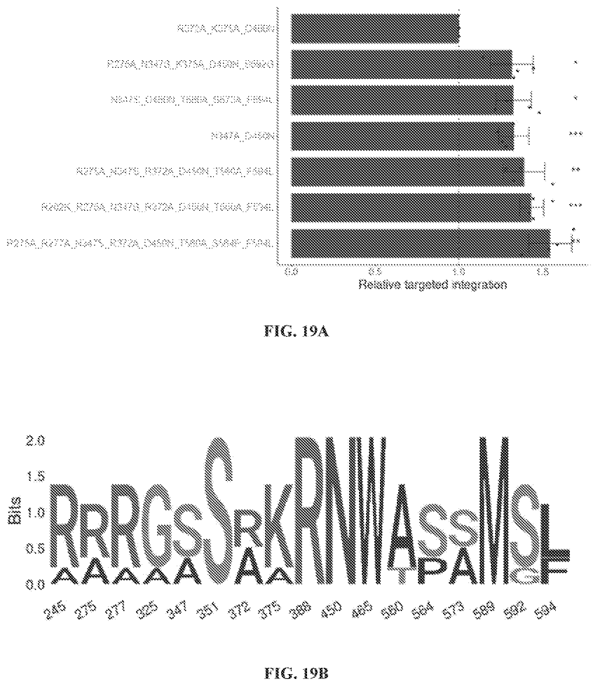

First, a random selection of 96 variants was performed and best performing variants were screened separately (FIG. 18). A summary of best PB amino acid variants for high on-target insertion confirms the importance of mutations D450N, R372A and K375A; but highlights other important residues which contribute to increased targeted efficiency (FIG. 19B). The six PB variants with best on-target efficiencies were selected (FIG. 19A). The individual on-target activities were significantly improved compared to FiCAT (Cas9-hyPB R372A-K375A-D450N) with the following variants: N347A-D450N; N347S-D450N-T560A-S573A-F594L; R202K-R275A-N347S-R372A-D450N-T560A-F594L; R275A-N347S-K375A-D450N-S592G; R275A-N347S-R372A-D450N-T560A-F594L; and R275A-R277A-N347S-R372A-D450N-T560A-S564P-F594L (two-sided t-test).

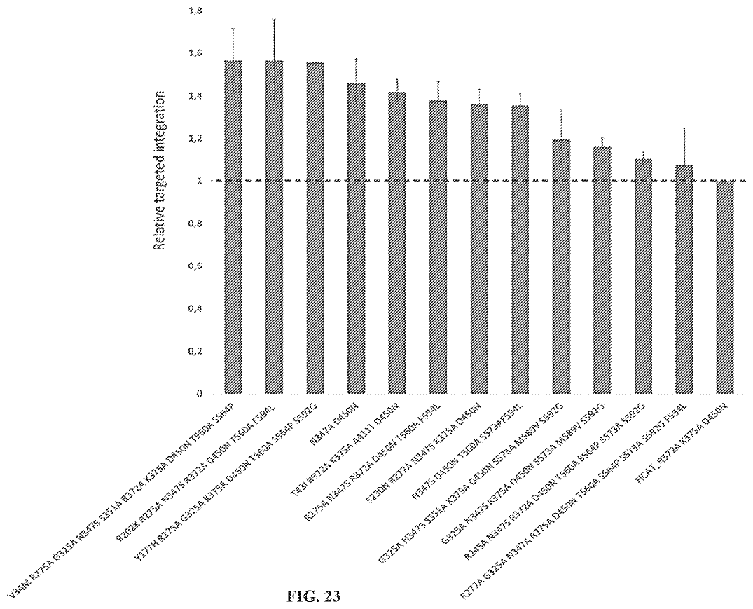

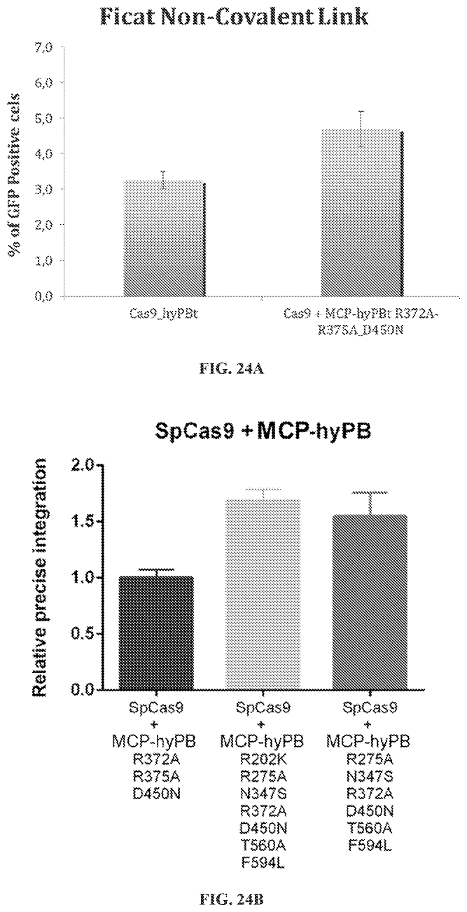

This experiment was repeated and confirmed (FIG. 22A). We also produced lentiviruses expressing bulk variants of each cycle and infected reporter cell line correcting its titer by the PB variants CN, demonstrating a similar increase of on-target efficiency over cycles (FIG. 22B). Single mutants were isolated from bulk variants after 4 and 5 cycles of cas9_PB library enrichment. Mutants were tested separately by transfecting on-target reporter cell line with FiCAT mutant, gRNA tcr1 and ½ GFP MC transposon. Best FiCAT mutants are shown in comparison with FiCAT R372A_K375A_D450N (FIG. 23). The individual on-target activities were significantly improved compared to FiCAT (Cas9-hyPB R372A-K375A-D450N) with the following variants: R202K-R275A-N347S-R372A-D450N-T560A-F594L; R245A-N347S-R372A-D450N-T560A-S564P-S573A-S592G; R275A-N347S-R372A-D450N-T560A-F594L; N347A-D450N; R277A-G325A-N347A-R375A-D450N-T560A-S564P-S573A-S592G-F594L; N347S-D450N-T560A-S573A-F594L; V34M-R275A-G325A-N347S-S351A-R372A-K375A-D450N-T560A-S564P; G325A-N347S-K375A-D450N-S573A-M589V-S592G; S230N-R277A-N347S-K375A-D450N; T43I-R372A-K375A-A411T-D450N; G325A-N347S-S351A-K375A-D450N-S573A-M589V-S592G; Y177H-R275A-G325A-K375A-D450N-T560A-S564P-S592G.

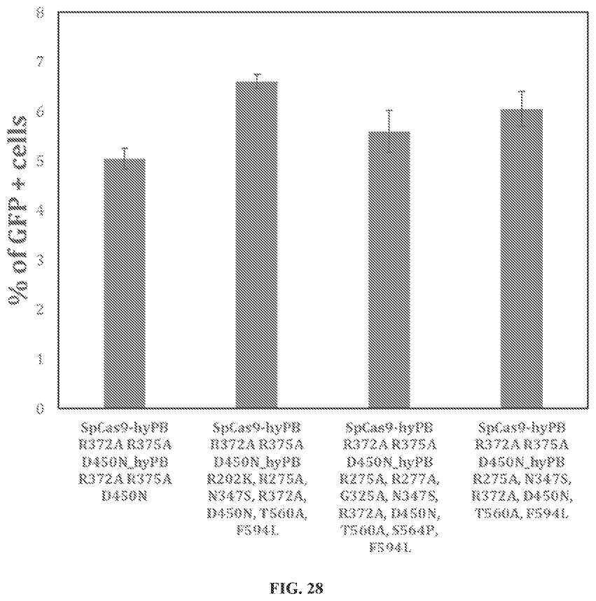

The superiority of mutants R202K-R275A-N347S-R372A-D450N-T560A-F594L, R275A-R277A-N347S-R372A-D450N-T560A-S564P-F594L and R275A-N347S-R372A-D450N-T560A-F594L compared to the mutant R372A-K375A-D450N was further demonstrated in triple fusion proteins comprising a SpCas9 and two hyPB (FIG. 28).

4.5. Results of Cas9 and hyPB Non-Covalent Linking

In addition to the covalent binding of Cas9 with hyPB R372A-R375A-D450N through a linker, we user the MS2-MCP system to link Cas9 and the fusion protein consisting of MCP protein and hyPB R372A-R375A-D450N through a modified gRNA containing a tetraloop of MS2 sequence binding the MCP protein.

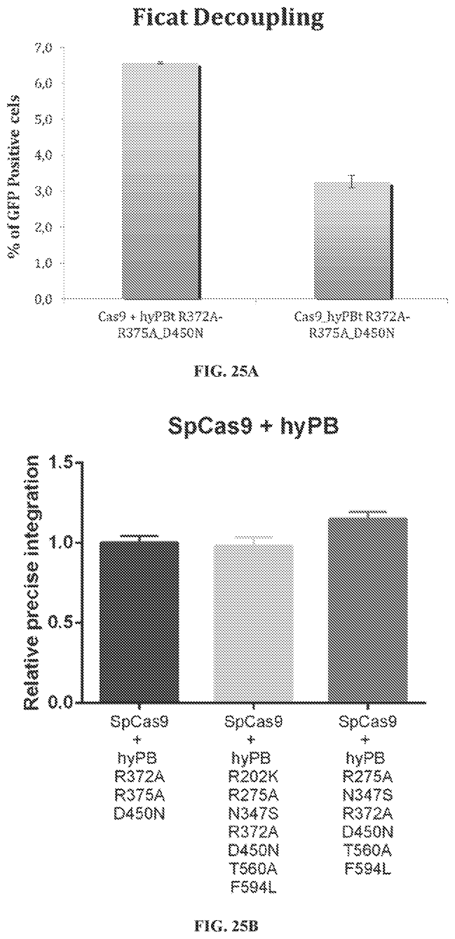

The combination of MCP-hyPB R372A-R375A-D450N fusion protein with Cas9 had an increased programmable insertion activity compared to Cas9-hyPB R372A-R375A-D450N fusion protein (FIG. 24A). In addition, we fused the MCP protein to other mutants of hyPB to perform programmable transposition in combination with SpCas9. Both variants used (R202K-R275A-N347S-R372A-D450N-T560A-F594L, and R275A-N347S-R372A-D450N-T560A-F594L) outperformed R372A-R375A-D450N (FIG. 24B).

4.6. Results of Cas9 and hyPB Decoupling for Programmable Transposition

We also tried the performance of SpCas9 with hyPB R372A-R375A-D450N without a linker, nor the MS2-MCP system. We co-expressed in the same cells SpCas9 and hyPB R372A-R375A-D450N, and an increased programmable insertion activity was registered compared to Cas9-hyPB R372A-R375A-D450N fusion protein (FIG. 25A). We extended the number of hyPB mutant variant tested not fused to Cas9, but being expressed at the same time, and acting together to achieve the activity of programmable transposition (FIG. 25B).

### 4.7. Results of Co-Expression of Cas9-hyPB and MCP-hyPB Fusion Proteins

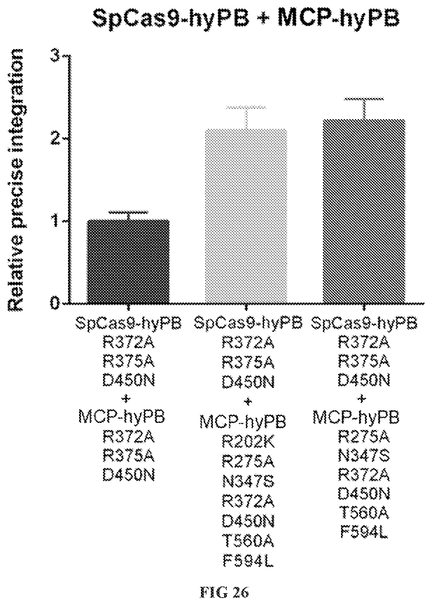

We co-transfected hyPB R372A-R375A-D450N mutant fused to MCP protein, and hyPB mutants fused to SpCas9, in order to obtain a dimeric version of the fusion with one of the monomers being non-covalently linked. Several hyPB mutants fused to SpCas9 were compared for specific target integration (FIG. 26).

4.8. Results of Co-Expression of Cas9-hyPB and hyPB Variants

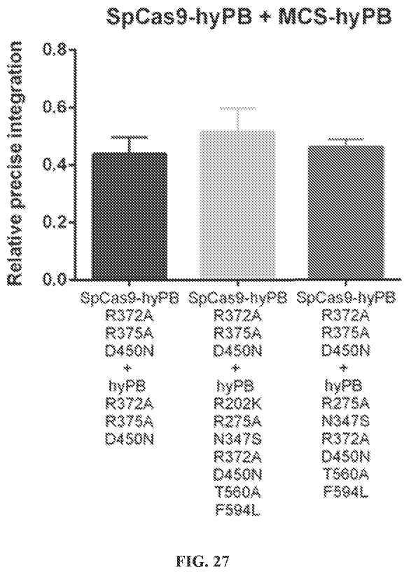

In a similar manner, we co-transfected the SpCas9 hyPB R372A-R375A-D450N fusion protein and hyPB mutants independently, in order to obtain a dimeric version of the fusion with one of the monomers not linked (FIG. 27).

### MATERIAL AND METHODS

### Lentivirus Production, Concentration and Titering

To produce virus, cells got co-transfected with pSICO (GFP) or pLV-Lama2 (Lama2) and pmd2.g (VSVG=envelope) and pax2 (containing packaging proteins, including IN) and sometimes with a plasmid just containing the wt-Integrase to rescue the non-infective Integrases. First 6×10⁵ HEK392T cells (passage 8) were seeded in a well of a 6-well tissue plat and incubated overnight. 5 h before starting the virus production, the media of cells was changed and on each well to 1.7 mL media containing 1:1000 CD (Chloroquine diphosphate; Stock=25 mM). The plasmids were infected in a molar ratio 1.6:1.32:0.72:3.32 (pSICO:pax2:VSVG:wtIN-rescue). PEI (Polyethylenimine; Stock=1 mg/mL) was used as transfection reagent, while 3 μL PEI was used for lug of total DNA used for transfection. DNA was diluted in 83 μL Opti-MEM (Thermo Scientific; #31985062) and PEI in another 83 μL. After mixing both solutions, they were incubated for 15-20 min at room temperature. Each transfection mix was added dropwise to the cells with the CD-Medium. Cells were incubated overnight. Media was changed by the next day and 2.5 mL fresh media was added. The next day the supernatant of cells was centrifuged for 5 min at 1000 rpm and passed through a 45 μm filter. Ultracentrifugation for 90 minutes at 19.500 rpm at 4° C. and resuspended overnight at 1:100 of the original media volume. The supernatant was stored at −80° C.

To determine the virus titer, HEK293T cells were infected with the produced virus and the amount of GFP positive cells was counted, since GFP was encoded on the viral packaging sequence. Therefore, 75000 HEK393T cells were seeded per well of a 6-well plate. Cells were infected with a mix of 1 mL media containing 1:1000 Polybrene and 500 μL previously produced Virus supernatant (1:3). The media was changed by the next day. The following day, the media was aspirated, and cells were detached using 200 μL Trypsin. The reaction was stopped adding 800 μL normal media and analyzed by cell cytometry. In the case of non-fluorescent Lama2 payload, gDNA was extracted from cells and qPCR was done to determine copy number for payload versus genomes.

### In Vivo Lama2 Delivery

Animal experimentation procedures were approved by the Animal Experimentation Ethic Committee of Barcelona Biomedical Research Park. C57BL/6J or Lama2 model dj2y/dj2y, 8-10 weeks old were used for this study. The animals were purchased from Jackson Laboratories, male and female were used without distinction.

Donor mice were sacrificed at 8 weeks of age and bone marrow cells were collected from hind limb bones; Lineage negative depletion was performed using Mouse Lineage Cell Depletion Kit (Miltenyi Biotec) following manufacturer instruction; and cells were transduced with Lentivirus overnight in Stem cell culture media with appropriate stimulating factors (StemCell Technologies). These cells were then harvested and transplanted into recipient mice (after conditioning by radiation) by retro-orbital injection.

Blood and tibialis muscle were collected from treated mice, 4 weeks after transplantation. Lysis Buffer (Thermo Fisher Scientific) was used to process blood samples following manufacturer instruction before FACS analysis. Tibialis muscles were minced and further processed with liberase/dispase digestion to isolate infiltrated cells before FACS analysis.

Grip strength assay was performed using a Bioseb device according to manufacturer instruction 6 and 12 weeks after treatment.

hyPB mRNA was produced with RiboMAX Large Scale RNA Production Systems-T7 (Promega) following manufacturer's instructions. Rosa26 gRNA (25) was purchased from IDT. hyPB mRNA, gRNA targeting Rosa26 and PB512-B or Lama2 transposon were injected via retro-orbital in a 1:2.5:2.5 ratio. A total of 55 μg of nucleic acids were complexed to; In vivo-JetPEI (Polyplus transfection) at NP ratio 7. Animals were euthanized 10 days after-injection and the liver was isolated and homogenized. Genomic DNA and RNA was extracted from liver samples. Transposon relative Copy number to Tfrc endogenous gene and transgene relative expression were obtained by qPCR, RT-qPCR or Elisa test respectively.

Imaging of luciferase expression was performed at different timepoints after FiCAT-gRNA-transposon or transposon control administration with IVIS spectrum imaging system (Caliper Life Sciences). Images were taken 5 min after intraperitoneal injection of D-Luciferin potassium salt (Gold Biotechnology) according to the manufacturer's instructions.

gDNA and RNA Harvest from Liver

Genomic DNA extraction was performed according to the DNeasy® Blood & Tissue Kit Protocol (QIAGEN®). Liver tissue was homogenized in PBS (phosphate-buffered saline). 20 μL of Proteinase K (provided by the kit) was added together with 200 μL of Buffer AL. After vortexing, the samples were incubated at 56° C. for 10 min. After the addition of 200 μL of ethanol (96-100%) and brief vortexing, the mixture was transferred into a Dneasy Mini spin column placed in a 3 mL collection tube and centrifuged at 8000 rpm for 1 min. The spin column was moved to a new 2 mL collection tube and 500 μL of Buffer AW1 was added. Tubes were centrifuged at 8000 rpm for 1 min. This washing step was repeated for Buffer AW2 (centrifugation of 3 min). Then, the spin was transferred to a new 1.5 mL microcentrifuge tube and 200 μL of Buffer AE was added to the center of the spin column membrane to elute the DNA by letting the tube stand for 1 min and it was followed by a centrifugation of 1 min at 8000 rpm. Concentration was measured with a NanoDrop One® (ThermoFisher Scientific).

qPCR and RT-qPCR

25 ng/μL and 10 ng/μL dilutions of genomic DNA samples were analyzed by qPCR. 5 μL of each dilution were mixed with 4.4 μL PowerUP SYBR Green MasterMix (Fisher Scientific) and 0.3 μL oligo forward and reverse. Oligos targeting Lama2, RFP and TfrC endogenous gene were used (SEQ ID NO: 100-105

Retrotranscription of mRNA to cDNA was performed using High-Capacity RNA-to-cDNA kit (ThermoFisher Scientific). 1 μg mRNA samples were mixed with 10 μL Buffer and 1 μL Enzyme mix in a 20 μL reaction. Reaction was incubated 2 h at 37° C. and inactivated for 10 min at 80° C. RT product was diluted 10 times and qPCR was performed mixing 5 μL of sample with 4.4 μL PowerUP SYBR Green MasterMix (Fisher Scientific) and 0.3 μL oligo fw and rv. Oligos targeting Lama2, RFP and GAPDH endogenous gene were used (SEQ ID NO: 102-107)

### Enzyme Linked Immunosorbent Assay (ELISA)

Whole blood was centrifuged at 4° C. 10 min 1200×g in a table top centrifuge. Plasma was separated and stored at 80° C. until ELISA assay was performed. Human Laminin subunit alpha-2 (LAMA-2) ELISA kit (Cusabio) was used. 20 μL of plasma was diluted with 80 μL of Sample buffer. Diluted samples were placed in the plate and incubated for 2 h. Wells were aspirated but not washed and 100 μL of Biotin-antibody was added. Plate was incubated for 1 h and washed 3 times with a wash buffer. 100 μL of HRP-avidin were added to each well and incubated at 37° C. 1 h. A washing step of 5 washed was performed carefully. 90 μL of TMB substrate was added and plate was incubated 20 min at 37° C. 50 μL of Stop Solution were added, plate was gently tapped and optical density was determined within 5 min at 450 nm in a Plate Reader.

### Fluorescent Activated Cytometry Analysis (FACS)

Circulating white blood cells and infiltrated cells in the muscle were isolated before emGFP expression was measured at (BD LSR Fortessa; BD Biosciences. Blue 488 nm laser with 530/30 filter and Yellow Green 561 nm laser with 610/20 filter).

1. A composition comprising:
a) a first protein comprising or consisting of a site-specific DNA binding protein capable of binding and cleaving a target nucleic acid sequence; or a nucleic acid construct encoding said first protein;
b) a second protein comprising or consisting of a transposase; or a nucleic acid construct encoding said second protein; and
c) a nucleic acid construct comprising a transgene encoding laminin-α2 protein or a functional variant or fragment thereof.

2. The composition according to claim 1, wherein the first and second proteins of a) and b) are fused together.

3. The composition according to claim 1, wherein said nucleic acid construct of c) comprises a promoter selected from the group consisting of: CMV promoter of SEQ ID NO: 76, CAG promoter of SEQ ID NO: 77, EF1-alpha promoter of SEQ ID NO: 78, SV-40 promoter of SEQ ID NO: 79 and EalbAAT promoter of SEQ ID NO: 80.

4. The composition according to claim 1, wherein said nucleic acid construct of c) comprises a splice acceptor of SEQ ID NO: 81.

5. The composition according to claim 1, wherein said nucleic acid construct of c) comprises a poly(A) signal sequence.

6. The composition according to claim 1, wherein said nucleic acid construct of c) is flanked by inverted terminal repeats (ITR).

7. The composition according to claim 1, wherein said nucleic acid construct of c) is comprised in a vector selected from the group consisting of: plasmid vector, a minicircle vector, a doggy bone DNA donor vector, a lentivirus vector and retrovirus vector.

8. The composition according to claim 1, wherein said site-specific DNA binding protein is RNA-guided nuclease comprising a Cas protein, and wherein said composition further comprises a guide RNA including a complementary sequence to a target nucleic acid sequence for integrating said LAMA2 transgene in a specific site of a genome of cell.

9. The composition according to claim 8, wherein the guide RNA comprises any one of SEQ ID NOs: 90 to 97.

10. The composition according to claim 1, wherein said transposase is a modified hyperactive Piggybac transposase or Sleepy Beauty transposase.

11. The composition according to claim 10, wherein said hyperactive PiggyBac transposase is a modified hyperactive PiggyBac transposase comprising at least one mutation of amino acid selected from the group consisting of: V34, T43, Y177, M194, R202, S230, R245, R275, R277, G325, S351, N347, R372, K375, R376, E377, E380, A411, D450, T560, S564, S573, M589, S592, and F594, said position number corresponding to the amino acid number of unmodified hyperactive Piggybac of SEQ ID NO: 9.

12. The composition according to claim 10, wherein said hyperactive PiggyBac transposase is a modified hyperactive PiggyBac transposase comprising at least one mutation of amino acid selected from the group consisting of: M194, R245, R275, R277, G325, R372, K375, R376, E377, E380, D450 and S573, said position number corresponding to the amino acid number of unmodified hyperactive Piggybac of SEQ ID NO: 9.

13. The composition according to claim 1, wherein said transposase is fused N-terminally to said site-specific DNA binding protein by a linker.

14. The composition according to claim 1, wherein said composition is packaged within a nanoparticle.

15. (canceled)

16. An engineered cell, wherein the engineered cell comprises a nucleic acid integrated within its genome, said nucleic acid comprising a transgene encoding laminin-α2 protein flanked by operational sequences for integrase- and/or transposase-mediated gene insertion.

17. The engineered cell according to claim 16, wherein the operational sequences flanking the transgene comprise or consists of SEQ ID NO: 88 and 89.

18. The engineered cell according to claim 16, wherein the inter-ITR size is at least 300 bp.

19. The engineered cell according to claim 16, wherein the transgene encodes the full-length laminin-α2 protein.

20.-21. (canceled)

22. The composition according to claim 13, wherein said linker is a peptidic linker comprising GGS, XTEN or FOKI.

23. A method for treating merosin-deficient congenital muscular dystrophy type 1A (MDC1A) in a subject in need thereof, comprising administering to said subject a therapeutically effective amount of the composition according to claim 1.
